(19) United States
(12) Patent Application Publication
(10) Pub. No.: US 2025/0340887 A1
(43) Pub. Date: Nov. 6, 2025

(54) PRODUCTS AND METHODS FOR TREATING DISEASES OR CONDITIONS ASSOCIATED WITH MUTANT OR PATHOGENIC KCNQ3 EXPRESSION

(71) Applicants: RESEARCH INSTITUTE AT NATIONWIDE CHILDREN'S HOSPITAL, Columbus, OH (US); THE TRUSTEES OF COLUMBIA UNIVERSITY IN THE CITY OF NEW YORK, New York, NY (US)

(72) Inventors: Scott Quenton Harper, Powell, OH (US); Wayne N. Frankel, Katonah, NY (US); Tristan T. Sands, New York, NY (US)

(21) Appl. No.: 18/872,100

(22) PCT Filed: Jun. 8, 2023
(86) PCT No.: PCT/US2023/068116

§ 371 Date: Dec. 5, 2024

Related U.S. Application Data
(60) Provisional application No. 63/350,279, filed on Jun. 8, 2022.

(51) Int. Cl. C12N 15/113; A61P 25/08; C12N 15/86

(52) U.S. Cl. CPC C12N 15/1138; A61P 25/08; C12N 15/86; C12N 2310/141; C12N 2750/14143; C12N 2830/008

(57) ABSTRACT

Disclosed herein are products, methods, and uses for treating, ameliorating, or delaying the progression of, and/or preventing seizures, an epileptic disease or disorder, an intellectual or developmental disability, autism, or an autism spectrum disorder associated with mutant or pathogenic Potassium Channel, Voltage Gated KQT-Like Subfamily Q, Member 3 (KCNQ3) expression. More particularly, disclosed herein are RNA interference-based products, methods, and uses for reducing or inhibiting the expression of the KCNQ3 gene and its resulting mRNA and/or protein. Even more particularly, the disclosure provides microRNA (miRNA) for reducing or inhibiting the expression of KCNQ3 and methods of using said miRNA to reduce or inhibit mutant or pathogenic KCNQ3 expression in cells and/or in cells of a subject having a genetic mutation in the KCNQ3 gene which results in disease symptoms including, but not limited to, seizures, epilepsy, intellectual and/or developmental disability, autism, or an autism spectrum disorder. Such disease symptoms, in some aspects, result from developmental and epileptic encephalopathy (DEE) attributed to various mutations in the KCNQ3 gene which result in the expression of various mutant or pathogenic forms of the KCNQ3 protein.

### INCORPORATION BY REFERENCE OF THE SEQUENCE LISTING

This application contains, as a separate part of disclosure, a Sequence Listing in computer-readable form (Filename: 57884_Seqlisting.XML; Size: 92,046 bytes; Created: Jun. 1, 2023) which is incorporated by reference herein in its entirety.

### FIELD

This disclosure relates to the field of the treatment of diseases associated with the mutant or pathogenic expression of the Potassium Channel, Voltage Gated KQT-Like Subfamily Q, Member 3 (KCNQ3) gene and the resulting protein. More particularly, the disclosure provides RNA interference-based products, methods, and uses for treating, ameliorating, delaying the progression of, and/or preventing diseases or conditions associated with the mutant or pathogenic expression of KCNQ3 protein resulting from one or more mutant forms of the KCNQ3 gene. Specifically, the disclosure provides products and methods for reducing or inhibiting the expression of one or more mutant or pathogenic forms of KCNQ3 by interfering with KCNQ3 gene expression mainly by binding with messenger RNA (mRNA) in the cell cytoplasm. More specifically, the disclosure provides microRNA (miRNA) for reducing or inhibiting the mutant or pathogenic expression of KCNQ3 and methods of using said miRNA to reduce or inhibit mutant or pathogenic KCNQ3 expression in cells and/or in a subject having neuronal excitability or disease symptoms resulting from such mutant or pathogenic KCNQ3 expression. Such disease symptoms include, but are not limited to, seizures, epilepsy, intellectual and/or developmental disability, autism, or an autism spectrum disorder. Such disease symptoms, in some aspects, result from developmental and epileptic encephalopathy (DEE) attributed to various mutations in the KCNQ3 gene which result in the expression of various mutant or pathogenic forms of the KCNQ3 protein.

### BACKGROUND

Standard pharmacotherapy is generally ineffective in children with Developmental and Epileptic Encephalopathies (DEE), despite the fact that >30% of cases are now precisely genetically diagnosed as de novo single gene variants (Stefanski et al., Epilepsia, 2021. 62(1): p. 143-151). About 40% of DEE genes with known pathogenic variants appear to require expression of a defective gene product (i.e. gain-of-function or dominant-negative) as opposed to encoding a partial, e.g. haploinsufficiency, or complete expression loss, and these tend to encode more severe disease (Wang et al. (2021) Neurobiol Dis 148: 105220). In the face of this challenge, and given recent and rapidly advancing progress in gene therapy technologies, and because of the exquisite specificity that gene therapy has for genetic lesions, RNAi technology provides great promise in patients who carry gain-of-function variants and who do not have many other options for effective therapy.

The disclosure provides products, compositions, and methods for an RNAi approach to decrease the expression of a pathogenic variant (KCNQ3-R230H) responsible for a form of DEE. This approach has been reduced to practice using a mouse model expressing the orthologous genotype (i.e. Kcnq3R231^{H/+}). Because mice that completely lack Kcnq3 from conception are only very mildly impaired with respect to overt clinical phenotypes or seizures (Soh et al. (2014) J Neurosci. 34: 5311-21), RNAi constructs were developed to target both mutant and wildtype copies of Kcnq3 mRNA. Using this approach to reduce wildtype Kcnq3 mRNA would have little or no detrimental effect in unaffected subjects, whereas reduction of the mutant Kcnq3 mRNA would significantly diminish phenotypic features in subjects that model or suffer from the human disease.

RNA interference (RNAi) is a mechanism of gene regulation in eukaryotic cells that has been considered for the treatment of various diseases. RNAi refers to post-transcriptional control of gene expression mediated by microRNAs (miRNAs). The miRNAs are small (21-25 nucleotides), noncoding RNAs that share sequence homology and base-pair with 3′ untranslated regions of cognate messenger RNAs (mRNAs). The interaction between the miRNAs and mRNAs directs cellular gene silencing machinery to prevent the translation of the mRNAs. The RNAi pathway is summarized in Duan (Ed.), Section 7.3 of Chapter 7 in Muscle Gene Therapy, Springer Science+Business Media, LLC (2010).

As an understanding of natural RNAi pathways has developed, researchers have designed artificial miRNAs for use in regulating expression of target genes for treating disease. As described in Section 7.4 of Duan, supra, artificial miRNAs can be transcribed from DNA expression cassettes. The miRNA sequence specific for a target gene is transcribed along with sequences required to direct processing of the miRNA in a cell. Viral vectors, such as adeno-associated virus (AAV) have been used to deliver miRNAs to muscle and the brain and nervous system [Fechner et al., J. Mol. Med., 86: 987-997 (2008)].

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hardy virus. It easily withstands the conditions used to inactivate adenovirus (56° to 65° C. for several hours), making cold preservation of AAV less critical. AAV may even be lyophilized. Finally, AAV-infected cells are not resistant to superinfection.

Care of patients suffering from DEE is limited to therapies that attempt to address the symptoms of the disorder, usually with limited results. Patients are seen by physical and occupational therapists, speech & language pathologists, and developmental specialists for their neurodevelopmental delay and autistic symptoms. In addition, patients may be treated with medications to address behavioral problems, sleep problems, and/or seizures. Despite these attempts, patients remain substantially impaired, non-verbal, and ultimately unable to care for themselves when they reach adulthood. The development of products and methods for effective disease modifying therapy for treating forms of DEE associated with pathogenic variants of the KCNQ3 gene, therefore, represents a critical unmet need.

### SUMMARY

The disclosure provides products, methods, and uses for reducing or inhibiting KCNQ3 gene expression, and ultimately interfering with translation of mutant or pathogenic forms of KCNQ3 for treating, ameliorating, delaying the progression of, and/or preventing seizures, epilepsy, intellectual and/or developmental disability, autism, or an autism spectrum disorder associated with mutant or pathogenic KCNQ3 expression including, but not limited to, developmental and epileptic encephalopathy (DEE). The disclosure provides products, methods, and uses for reducing or inhibiting KCNQ3 gene expression of mutant or pathogenic variants of KCNQ3. Such mutations include various missense mutations, gain-of function-mutations, and any mutations which alter the expression of KCNQ3 protein. In some aspects, such mutations in the KCNQ3 gene are known mutant or pathogenic variants including, but not limited to, KCNQ3-R230C, KCNQ3-R230H, KCNQ3-R230S, and/or KCNQ3-R227Q.

The disclosure provides nucleic acids designed to reduce or inhibit KCNQ3 expression or mutant or pathogenic expression of KCNQ3, viral vectors comprising the nucleic acids, compositions comprising the nucleic acids and vectors, methods for using these products for reducing or inhibiting and/or interfering with expression of a mutant or pathogenic KCNQ3 gene in a cell, and methods for treating or ameliorating disease in a subject suffering from a disease resulting from expression of a mutant or pathogenic variant of KCNQ3 including, but not limited to, KCNQ3-R230C, KCNQ3-R230H, KCNQ3-R230S, and or KCNQ3-R227Q.

The disclosure provides a nucleic acid encoding a Potassium Channel, Voltage Gated KQT-Like Subfamily Q, Member 3 (KCNQ3)-targeting microRNA (miRNA) comprising: (a) a nucleotide sequence comprising at least 90% identity to the sequence set forth in any one of SEQ ID NOs: 3-9; (b) the nucleotide sequence set forth in any one of SEQ ID NOs: 3-9; (c) a nucleotide sequence that encodes the RNA sequence set forth in any one of SEQ ID NOs: 17-23; or (d) a nucleotide sequence that specifically hybridizes to the KCNQ3 sequence set forth in any one of SEQ ID NOs: 24-30. Such nucleic acid, in some aspects, further comprises a promoter and/or enhancer. In some aspect, such promoter and/or enhancer is any of a U6 promoter and/or enhancer, a U7 promoter and/or enhancer, a tRNA promoter and/or enhancer, an H1 promoter and/or enhancer, a CMV promoter and/or enhancer, a minimal CMV promoter and/or enhancer, a T7 promoter and/or enhancer, an EF1-alpha promoter and/or enhancer, a minimal EF1-alpha promoter and/or enhancer, an unc45b promoter and/or enhancer, a CK1 promoter and/or enhancer, a CK6 promoter and/or enhancer, a CK7 promoter and/or enhancer, a CK8 promoter and/or enhancer, a ubiquitous promoter and/or enhancer, a neuronal-specific promoter and/or enhancer, or a brain-specific promoter and/or enhancer. In some aspects, the promoter and/or enhancer is U6. In some aspects, the nucleic acid comprises (a) a nucleotide sequence comprising at least 90% identity to the sequence set forth in any one of SEQ ID NOs: 10-16; or (b) the nucleotide sequence set forth in any one of SEQ ID NOs: 10-16. In some aspects, the brain-specific promoter and/or enhancer is human Synapsin1 (hSyn1), neuron-specific enolase (Nse), MeCP2, mDLX, mDLX5/6, or calmodulin-dependent kinase II (CaMKII or Camk2a).

The disclosure provides an adeno-associated virus comprising a nucleic acid encoding a Potassium Channel, Voltage Gated KQT-Like Subfamily Q, Member 3 (KCNQ3)-targeting microRNA (miRNA) comprising: (a) a nucleotide sequence comprising at least 90% identity to the sequence set forth in any one of SEQ ID NOs: 3-9; (b) the nucleotide sequence set forth in any one of SEQ ID NOs: 3-9; (c) a nucleotide sequence that encodes the RNA sequence set forth in any one of SEQ ID NOs: 17-23; or (d) a nucleotide sequence that specifically hybridizes to the KCNQ3 sequence set forth in any one of SEQ ID NOs: 24-30. Such nucleic acid, in some aspects, further comprises a promoter and/or enhancer. In some aspect, such promoter and/or enhancer is any of a U6 promoter and/or enhancer, a U7 promoter and/or enhancer, a tRNA promoter and/or enhancer, an H1 promoter and/or enhancer, a CMV promoter and/or enhancer, a minimal CMV promoter and/or enhancer, a T7 promoter and/or enhancer, an EF1-alpha promoter and/or enhancer, a minimal EF1-alpha promoter and/or enhancer, an unc45b promoter and/or enhancer, a CK1 promoter and/or enhancer, a CK6 promoter and/or enhancer, a CK7 promoter and/or enhancer, a CK8 promoter and/or enhancer, a ubiquitous promoter and/or enhancer, a neuronal-specific promoter and/or enhancer, or a brain-specific promoter and/or enhancer. In some aspects, the promoter and/or enhancer is U6. In some aspects, the nucleic acid comprises (a) a nucleotide sequence comprising at least 90% identity to the sequence set forth in any one of SEQ ID NOs: 10-16; or (b) the nucleotide sequence set forth in any one of SEQ ID NOs: 10-16. In some aspects, the brain-specific promoter and/or enhancer is human Synapsin1 (hSyn1), neuron-specific enolase (Nse), MeCP2, mDLX, mDLX5/6, or calmodulin-dependent kinase II (CaMKII or Camk2a). In some aspects, the adeno-associated virus lacks rep and cap genes. In some aspects, the virus is a recombinant AAV (rAAV) or a self-complementary recombinant AAV (scAAV). In some aspects, the virus is AAV1, AAV2, AAV3, AAV4, AAV5, AAV6, AAV7, AAV8, AAV9, AAV.rh74, AAV.rh8, AAV.rh10, AAV11, AAV12, AAV13, AAV-anc80, AAV-B1, AAV.PHP.EB, or AAVv66. In some aspects, the virus is AAV9.

The disclosure provides a nanoparticle, extracellular vesicle, or exosome comprising a nucleic acid encoding a Potassium Channel, Voltage Gated KQT-Like Subfamily Q, Member 3 (KCNQ3)-targeting microRNA (miRNA) comprising: (a) a nucleotide sequence comprising at least 90% identity to the sequence set forth in any one of SEQ ID NOs: 3-9; (b) the nucleotide sequence set forth in any one of SEQ ID NOs: 3-9; (c) a nucleotide sequence that encodes the RNA sequence set forth in any one of SEQ ID NOs: 17-23; or (d) a nucleotide sequence that specifically hybridizes to the KCNQ3 sequence set forth in any one of SEQ ID NOs: 24-30. Such nucleic acid, in some aspects, further comprises a promoter and/or enhancer. In some aspect, such promoter and/or enhancer is any of a U6 promoter and/or enhancer, a U7 promoter and/or enhancer, a tRNA promoter and/or enhancer, an H1 promoter and/or enhancer, a CMV promoter and/or enhancer, a minimal CMV promoter and/or enhancer, a T7 promoter and/or enhancer, an EF1-alpha promoter and/or enhancer, a minimal EF1-alpha promoter and/or enhancer, an unc45b promoter and/or enhancer, a CK1 promoter and/or enhancer, a CK6 promoter and/or enhancer, a CK7 promoter and/or enhancer, a CK8 promoter and/or enhancer, a ubiquitous promoter and/or enhancer, a neuronal-specific promoter and/or enhancer, or a brain-specific promoter and/or enhancer. In some aspects, the promoter and/or enhancer is U6. In some aspects, the nucleic acid comprises (a) a nucleotide sequence comprising at least 90% identity to the sequence set forth in any one of SEQ ID NOs: 10-16; or (b) the nucleotide sequence set forth in any one of SEQ ID NOs: 10-16. In some aspects, the brain-specific promoter and/or enhancer is human Synapsin1 (hSyn1), neuron-specific enolase (Nse), MeCP2, mDLX, mDLX5/6, or calmodulin-dependent kinase II (CaMKII or Camk2a).

The disclosure provides a composition comprising (a) any one or more of the nucleic acids as described herein above or throughout the disclosure; (b) any one or more of the adeno-associated viruses as described herein above or throughout the disclosure; or (c) any one or more of the nanoparticles, extracellular vesicles, or exosomes as described herein above or throughout the disclosure; and a pharmaceutically acceptable carrier.

The disclosure provides a method of reducing or inhibiting and/or interfering with expression of a Potassium Channel, Voltage Gated KQT-Like Subfamily Q, Member 3 (KCNQ3) gene or a variant thereof in a cell comprising contacting the cell with (a) any one or more of the nucleic acids as described herein above or throughout the disclosure; (b) any one or more of the adeno-associated viruses as described herein above or throughout the disclosure; or (c) any one or more of the nanoparticles, extracellular vesicles, or exosomes as described herein above or throughout the disclosure; or (d) a composition as described herein above or throughout the disclosure.

The disclosure provides a method of treating a subject having a KCNQ3 mutation that results in the expression of a mutant or pathogenic form of KCNQ3 comprising administering to the subject an effective amount of (a) any one or more of the nucleic acids as described herein above or throughout the disclosure; (b) any one or more of the adeno-associated viruses as described herein above or throughout the disclosure; or (c) any one or more of the nanoparticles, extracellular vesicles, or exosomes as described herein above or throughout the disclosure; or (d) a composition as described herein above or throughout the disclosure. In some aspects, the mutation is a missense mutation or a gain-of-function mutation. In some aspects, the mutation is point mutation, a frameshift mutation, a base substitution, a deletion, or an insertion, or a combination of any of these mutations. In some aspects, the mutation is the mutation is a base substation, deletion, or insertion, or a combination of any thereof. In some aspects, the mutation is any one or more mutations in the KCNQ3 gene resulting in the substitution of R230C, R230H, R230S, and/or R227Q of the KCNQ3 polypeptide. In some aspects, the mutation results in the subject suffering from any of a variety of symptoms associated with the mutant or pathogenic expression of KCNQ3. In some aspects, the subject suffers from seizures, an epileptic disease or disorder, an intellectual or developmental disability, autism, or an autism spectrum disorder associated with mutant or pathogenic KCNQ3 expression.

The disclosure provides a method of treating or ameliorating a subject suffering from seizures, an epileptic disease or disorder, an intellectual or developmental disability, autism, or an autism spectrum disorder associated with mutant or pathogenic KCNQ3 expression comprising administering to the subject an effective amount of (a) any one or more of the nucleic acids as described herein above or throughout the disclosure; (b) any one or more of the adeno-associated viruses as described herein above or throughout the disclosure; or (c) any one or more of the nanoparticles, extracellular vesicles, or exosomes as described herein above or throughout the disclosure; or (d) a composition as described herein above or throughout the disclosure. In some aspects, the subject suffers from or is at risk of suffering from developmental and epileptic encephalopathy (DEE). In some aspects, the subject suffers from any mutation in the KCNQ3 gene. Such mutations include, but are not limited to, missense mutations and gain-of-function mutations. In some aspects, the subject suffers from a mutation in the KCNQ3 gene, wherein the mutation is any one or more mutations in the KCNQ3 gene resulting in the substitution of R230C, R230H, R230S, and/or R227Q of the KCNQ3 polypeptide.

The disclosure provides a use of (a) any one or more of the nucleic acids as described herein above or throughout the disclosure; (b) any one or more of the adeno-associated viruses as described herein above or throughout the disclosure; or (c) any one or more of the nanoparticles, extracellular vesicles, or exosomes as described herein above or throughout the disclosure; or (d) a composition as described herein above or throughout the disclosure for the preparation of a medicament for reducing or inhibiting expression of a Potassium Channel, Voltage Gated KQT-Like Subfamily Q, Member 3 (KCNQ3) gene or a variant thereof in a cell.

The disclosure provides a use of (a) any one or more of the nucleic acids as described herein above or throughout the disclosure; (b) any one or more of the adeno-associated viruses as described herein above or throughout the disclosure; or (c) any one or more of the nanoparticles, extracellular vesicles, or exosomes as described herein above or throughout the disclosure; or (d) a composition as described herein above or throughout the disclosure for treating or ameliorating seizures, an epileptic disease or disorder, an intellectual or developmental disability, autism, or an autism spectrum disorder associated with mutant or pathogenic KCNQ3 expression. In some aspects, the seizures, epileptic disease or disorder, the intellectual or developmental disability, autism, or the autism spectrum disorder associated with mutant or pathogenic KCNQ3 expression is developmental and epileptic encephalopathy (DEE). In some aspects, the seizures, epileptic disease or disorder, the intellectual or developmental disability, autism, or the autism spectrum disorder associated with mutant or pathogenic KCNQ3 expression results from any mutation in the KCNQ3 gene. In some aspects, the mutation is any one or more mutations in the KCNQ3 gene resulting in the substitution of R230C, R230H, R230S, and/or R227Q of the KCNQ3 polypeptide.

The disclosure provides a use of (a) any one or more of the nucleic acids as described herein above or throughout the disclosure; (b) any one or more of the adeno-associated viruses as described herein above or throughout the disclosure; or (c) any one or more of the nanoparticles, extracellular vesicles, or exosomes as described herein above or throughout the disclosure; or (d) a composition as described herein above or throughout the disclosure for the preparation of a medicament for treating or ameliorating seizures, an epileptic disease or disorder, an intellectual or developmental disability, autism, or an autism spectrum disorder associated with mutant or pathogenic KCNQ3 expression. In some aspects, the seizures, epileptic disease or disorder, the intellectual or developmental disability, autism, or the autism spectrum disorder associated with mutant or pathogenic KCNQ3 expression is developmental and epileptic encephalopathy (DEE). In some aspects, the seizures, epileptic disease or disorder, the intellectual or developmental disability, autism, or the autism spectrum disorder associated with mutant or pathogenic KCNQ3 expression results from any mutation in the KCNQ3 gene. In some aspects, the mutation is any one or more mutations in the KCNQ3 gene resulting in the substitution of R230C, R230H, R230S, and/or R227Q of the KCNQ3 polypeptide.

The disclosure provides a use of (a) any one or more of the nucleic acids as described herein above or throughout the disclosure; (b) any one or more of the adeno-associated viruses as described herein above or throughout the disclosure; or (c) any one or more of the nanoparticles, extracellular vesicles, or exosomes as described herein above or throughout the disclosure; or (d) a composition as described herein above or throughout the disclosure for reducing or inhibiting and/or interfering with expression of a Potassium Channel, Voltage Gated KQT-Like Subfamily Q, Member 3 (KCNQ3) gene or a variant thereof in a cell. In some aspects, the cell is in a subject. In some aspects, the variant of KCNQ3 results from any mutation in the KCNQ3 gene. In some aspects, the variant of the KCNQ3 results in the substitution of R230C, R230H, R230S, and/or R227Q of the KCNQ3 polypeptide.

In some aspects, the nucleic acid, AAV, nanoparticle, extracellular vesicle, exosome, or composition, or medicament of the disclosure is formulated for intracerebroventricular injection, intrathecal injection, injection into the blood stream, aerosol administration, or oral administration.

Further aspects and advantages of the disclosure will be apparent to those of ordinary skill in the art from a review of the following detailed description, taken in conjunction with the drawings. It should be understood, however, that the detailed description (including the drawings and the specific examples), while indicating embodiments of the disclosed subject matter, are given by way of illustration only, because various changes and modifications within the spirit and scope of the disclosure will become apparent to those skilled in the art from this detailed description.

### BRIEF DESCRIPTION OF THE DRAWINGS

FIG. 1 shows the microRNA design parameters used for designing KCNQ3 miRNAs of the disclosure. Artificial microRNAs were designed using an algorithm as previously described (Wallace et al. (2017) Mol Ther Methods Clin. Dev. December 24, 8:121-130; Boudreau et al. (2011) RNA Interference Methods. Ed. S. Q. Harper. Humana Springer Press, 2011, pages 19-37). In brief, all microRNAs contain processing sites for the RNAse III enzymes Drosha and Dicer, yielding a mature, 22 nucleotide (nt) duplex RNA containing 2 nt 3′ overhangs on both strands. The antisense guide strand of the microRNAs become incorporated into the RNA-Induced Silencing Complex (RISC), where they direct cellular gene silencing machinery to cleave target mRNAs, in this case human KCNQ3 or rodent Kcnq3.

FIG. 2 shows the sequences and structures of miKCNQ3 constructs. Top left shows natural human mir30a sequence and structure. Artificial miRNAs incorporate some sequence and structural features from mir30a, but with modifications. In particular, the underlined mature mir30a and corresponding sense strand, located between the arrowheads on this figure, are replaced with 22 nucleotide sequences targeting conserved regions on human, mouse, and rat KCNQ3 open reading frames. All microRNAs were designed as DNA constructs, and cloned into the U6T6 plasmid using the indicated restriction enzymes (the SpeI site is ligated to an XbaI site located in the U6T6 polylinker). Top right shows the RNA sequence of miKCNQ3-A, with the mature duplex sequences located between the arrowheads. The gray arrowheads indicate cut sites for Drosha, while the black arrows show Dicer cut sites. Mismatches at positions 13 and 75 help facilitate proper Drosha processing, and the indicated mismatches were incorporated in each miRNA as indicated. Positions 35 and 53 are indicated for orientation. The antisense guide strands of all microRNAs (miKCNQ3 A-G) contain perfect 22 nt base-pairing homology with human, mouse, and rat KCNQ3/Kcnq3 sequences.

FIG. 3 shows the constructed luciferase reporter plasmids containing human KCNQ3 or rodent Kcnq3 sequences as the 3′ UTR of Renilla luciferase used to measure silencing of human or rodent KCNQ3. The reporter plasmid contained a second gene, Firefly luciferase, which was used as a normalization control.

FIG. 4 shows results of luciferase assay screens. The various KCNQ3 miRNA were cloned as 3′ UTR of Renilla luciferase. A second reporter, Firefly luciferase, was present on the same plasmid and served as a non-targeted transfection control. HEK293 cells were transfected with the indicated reporter plasmids along with U6.miKCNQ3 expression cassettes or the miGFP control. Luciferase assays showed that each of the miKCNQ3 microRNA of the disclosure were able to knockdown human and rodent KCNQ3/Kcnq3 transcripts. Luciferase assays identified miKCNQ3-A as a lead microRNA for initial experiments to knockdown human and rodent KCNQ3/Kcnq3 transcripts. Data shown are representative of three independent experiments for each indicated reporter construct. Error bars indicate S.E.M.

FIG. 5 shows a representative cluster of three spike-wave discharges (arrows) in an EEG of a Kcnq3^{R231H/+} adult mouse (bottom two traces) vs wildtype Kcnq3^{+/+} mice (top two traces). “FR” and “FL” refer to signal of bilateral electrodes situated over the right (R) or left (L) hemisphere in front of the Bregma line, to a reference electrode situated over the cerebellum. In characterizing clinically-relevant phenotypic features of the Kcnq3^{R231H/+} mice, it was determined that heterozygotes, but not wildtype littermates, have a type of generalized epilepsy in the form of frequent, spontaneous spike-wave discharges (SWD) in the electroencephalogram (EEG). It also was determined that heterozygotes have a significantly decreased threshold (increased susceptibility) to electrically-induced maximal seizures, compared to wildtype littermates, indicating a general propensity towards seizures. Each of these electroclinical features is quantitative and reproducible, representing strong and clinically-relevant endpoints against which to measure efficacy of new therapies.

FIG. 6A-D shows that the Kcnq3^{R231H/+} genotype does not cause growth delay in mouse pups (FIG. 6A) or alter Kcnq3 mRNA (FIG. 6B) or total protein (FIG. 6C) expression, but it may be associated with increased KCNQ3 protein at neuronal membranes (FIG. 6D).

FIG. 7A-B shows that there was a significant decrease in SWD incidence (FIG. 7A) and SWD average duration (FIG. 7B) in adult Kcnq3^{R231H/+} mice transduced with scAAV9-miKCNQ3 as neonates.

FIG. 8 shows a decrease in SWD incidence (top panel) and a decrease in SWD duration (bottom panel) in Kcnq3^{R231H/+} adult mice transduced with scAAV9-miKcnq3-A as neonates. Dotted lines show the same mice tested at both ages. The p-value shown is based on a 1-tailed Fisher Exact Test.

FIG. 9 shows that there was a significant decrease in Kcnq3 mRNA in adult Kcnq3^{R231H/+} mice transduced with scAAV9-miKCNQ3 as neonates. From mice that were previously treated with scAAV9-U6-miKcnq3a-eGFP or with scAAV9-eGFP control virus and assessed for EEG activity, qPCR data were generated to measure endogenous Kcnq3 mRNA, and endogenous Actb as an internal control. Exogenous eGFP mRNA was also measured, as a control for AAV9 transduction. The number of amplification cycles that differ between Kcnq3 and Actb (ΔCt, top panel), eGFP and Actb (ΔCt, middle panel), or Kcnq3-Actb-eGFP (ΔCt, bottom panel) were determined and plotted. FIG. 9 shows separate marker shapes or shading which reflect different biological replicates (technical replicates share a marker shape). Because eGFP is an internal control for transduced cells, statistical significance shown is between miKcnq3a and eGFP treated mice (bottom panel), using a least squares regression with non-parametric transformed data and a post-hoc Dunnett test. Leaving out one miKcnq3-treated heterozygote that still had some SWD, the fold-difference in mRNA expression between miKcnq3 vs. control virus treated heterozygotes was 6.78-fold.

FIG. 10A-B shows a Western blot (FIG. 10A) with densitometry quantification (FIG. 10B) measuring KCNQ3 level and a loading control, β-actin, in adult mouse brains after treatment with scAAV9-miKcnq3 as neonates. FIG. 10B provides results after quantification of KCNQ3/β-actin following densitometry. There was a significant decrease of KCNQ3 protein in adult mice that were transduced with scAAV9-miKcnq3 as neonates.

FIG. 11 shows the sequence of miKCNQ3A, including the mature duplex sequence and the guide strand (antisense) sequence.

FIG. 12 shows the sequence of miKCNQ3B, including the mature duplex sequence and the guide strand (antisense) sequence.

FIG. 13 shows the sequence of miKCNQ3C, including the mature duplex sequence and the guide strand (antisense) sequence.

FIG. 14 shows the sequence of miKCNQ3D, including the mature duplex sequence and the guide strand (antisense) sequence.

FIG. 15 shows the sequence of miKCNQ3E, including the mature duplex sequence and the guide strand (antisense) sequence.

FIG. 16 shows the sequence of miKCNQ3F, including the mature duplex sequence and the guide strand (antisense) sequence.

FIG. 17 shows the sequence of miKCNQ3G, including the mature duplex sequence and the guide strand (antisense) sequence.

### DETAILED DESCRIPTION

The disclosure provides a novel strategy to accomplish control of Potassium Channel, Voltage Gated KQT-Like Subfamily Q, Member 3 (KCNQ3) gene expression by binding with KCNQ3 messenger RNA (mRNA) post-transcriptionally and repressing or reducing or inhibiting KCNQ3 protein production because the expression of a variant or pathogenic form of KCNQ3 results from KCNQ3 gain-of-function mutations. Such KCNQ3 mutations, for example, known as KCNQ3 R230C/H/S and KCNQ3 R227Q mutations result in the production of a pathogenic form of KCNQ3 protein in the brain, which is known to cause seizures and epilepsy including, but not limited to, developmental and epileptic encephalopathy (DEE). Thus, in some aspects, the products and methods described herein are used in treating, ameliorating, delaying the progression of, and/or preventing seizures, an epileptic disease or disorder, an intellectual or developmental disability, neurodevelopmental disability (NDD), autism, or an autism spectrum disorder including, but not limited to, DEE.

The KCNQ3 gene belongs to a large family of genes that provide instructions for making potassium channels. These channels, which transport positively charged atoms (ions) of potassium into and out of cells, play a key role in a cell's ability to generate and transmit electrical signals. The specific function of a potassium channel depends on its protein components and its location in the body. Channels made with the KCNQ3 protein are active in nerve cells (neurons) in the brain, where they transport potassium ions out of cells. These channels transmit a particular type of electrical signal called the M-current, which prevents the neuron from continuing to send signals to other neurons. The M-current ensures that the neuron is not constantly active, or excitable. Potassium channels are made up of several protein components (subunits). Each channel contains four alpha subunits that form the hole (pore) through which potassium ions move. Four alpha subunits from the KCNQ3 gene can form a channel.

Some mutations in the KCNQ3 gene resulting in the mutant or pathogenic expression of KCNQ3 protein have been identified in people that suffer from DEE and/or seizures, an epileptic disease or disorder, an intellectual or developmental disability, neurodevelopmental disability (NDD), autism, or an autism spectrum disorder. For example, various mutations in the KCNQ3 gene, such as R230C, R230H, R230S, and R227Q, have each been reported to be a gain-of-function mutations in human patients (Sands et al., Ann. Neurol. 2019; 86: 181-92). Patients identified with such heterozygous KCNQ3 de novo variants (DNVs) exhibited developmental delays, autistic features, autism spectrum disorder, and epileptiform discharges or epileptic spikes (Sands et al., Ann. Neurol. 2019; 86: 181-92). A gain-of-function mutation is a type of mutation in which the altered gene product possesses a new molecular function or a new pattern of gene expression. Gain-of-function (GoF) mutations are almost always dominant or semi-dominant. The disclosure includes products and methods for treating such various KCNQ3 gene mutations and patients having such genetic mutations. Although only a limited number of mutations are known, the disclosure includes products and methods for treating any KCNQ3 gene mutations and patients having such genetic mutations which result in a mutated or pathogenic form of the KCNQ3 protein. In some aspects, such inherited mutations include the gain-of-function mutations (R230C, R230H, R230S, and R227Q of KCNQ3) which cause DEE. Sands et al. postulated that the R227Q or the R230C/S/H substitutions are predicted to selectively destabilize the resting (closed) conformation of the KCNQ3 voltage sensing domain (VSD), possibly explaining the observed GoF effects. Although the miRNA of the disclosure are not allele specific, the products and methods of the disclosure are designed to reduce or inhibit expression of mutant forms of the KCNQ3 gene which result in the expression of a mutated or pathogenic form of the KCNQ3 protein. This is because patients with normal KCNQ3 gene expression have no need for such therapeutic invention.

The disclosure provides the use of a mouse model as a mechanistic basis for studying the effects of miRNAs on the GoF by KCNQ3 R227 and R230 variants. The R231H variant in mice is equivalent to the R230 variant in humans. C57BL/6J and FVB/NJ mice were purchased from The Jackson Laboratory and maintained by brother-sister matings in the vivarium at Columbia University. Kcnq3^{R231H} mice were developed in the transgenic core at the Columbia Herbert Irving Comprehensive Cancer Center by using CRISPR/Cas9 mutagenesis with a donor oligonucleotide in C57BL/6J zygotes with the sgRNA 5′-GCAGGAUCUGCAGGAAGCGA-3′ (SEQ ID NO: 38) to change the Arg 231 CGC codon to CAC His and also to eliminate a PstI restriction enzyme site for convenient genotyping. Founder mice were crossed to wildtype C57BL/6J and thereafter backcrossed to wildtype C57BL/6J to maintain the line. For RNAi studies, Kcnq3R231^{H/+} heterozygous males were mated to wildtype FVB/NJ to make the F1 hybrid population segregating the Kcnq3^{R231H} mutation and used for viral injection, EEG testing, and assessment of mRNA and protein abundance (Sands et al., www.aesnet.org/abstractslisting/kcnq3-gain-of-function-mouse-model—electroclinical-and-behavioral-phenotype).

The disclosure provides products and methods designed to treat seizures, an epileptic disease or disorder, an intellectual or developmental disability, neurodevelopmental disability (NDD), autism, or an autism spectrum disorder resulting from the mutant or pathogenic expression of KCNQ3. The disclosure provides products and methods for preventing, treating or ameliorating conditions resulting from any mutations in the KCNQ3 gene which result in the mutant or pathogenic expression of KCNQ3. More specifically, the disclosure provides products and methods for preventing, treating or ameliorating conditions resulting from inherited and/or de novo missense mutations in the KCNQ3 gene. In some aspects, the condition or disease resulting from the mutant or pathogenic expression of KCNQ3 is DEE. In some aspects, such inherited and/or de novo mutations include the gain-of-function mutations (R230C, R230H, R230S, and R227Q of KCNQ3) which cause DEE. Thus, the products and methods of the disclosure are designed to treat diseases or disorders which result from any mutations in the KCNQ3 gene including, but not limited to, R230C, R230H, R230S, and/or R227Q mutations which result in the mutant or pathogenic expression of KCNQ3.

Inherited missense variants that result in a loss-of-function cause a dominantly inherited syndrome with seizures in newborns that respond to treatment and is outgrown in time and, thus, are not expected to benefit from the products and methods of the disclosure. Another form of DEE is caused by homozygous mutations that result in loss-of-function and this form is likewise not expected to benefit from treatment.

The KCNQ3 gene (Gene ID: 3786; ncbi.nlm.nih.gov/gene/3786) encodes a protein that functions in the regulation of neuronal excitability. The encoded protein forms an M-channel by associating with the products of the related KCNQ2 or KCNQ5 genes, which both encode integral membrane proteins. M-channel currents are inhibited by M1 muscarinic acetylcholine receptors and are activated by retigabine, a novel anti-convulsant drug. Defects in this KCNQ3 gene are a cause of benign familial neonatal convulsions type 2 (BFNC2), also known as epilepsy, benign neonatal type 2 (EBN2). Alternative splicing of this gene results in multiple transcript variants.

In some aspects, the nucleic acid encoding human KCNQ3 is set forth in the nucleotide sequence set forth in SEQ ID NO: 1. In some aspects, the amino acid sequence of human KCNQ3 is set forth in the amino acid sequence set forth in SEQ ID NO: 2. In various aspects, the methods of the disclosure also target isoforms and variants of the nucleotide sequence set forth in SEQ ID NO: 1. In some aspects, the variants comprise 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 1 In some aspects, the methods of the disclosure target isoforms and variants of nucleic acids comprising nucleotide sequences encoding the amino acid sequence set forth in SEQ ID NO: 2. In some aspects, the variants comprise 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to a nucleotide sequence that encodes the amino acid sequence set forth in SEQ ID NO: 2.

TABLE 1
KCNQ3 DNA and Amino Acid Sequences

| SEQ ID | |
|---|---|
| NO: | Sequence |
| 1 | atggggctcaaggcgcgcagggcggcgggggcggc |
| (KCNQ3 | tggcggcggcggcgacgggggcggcggaggcggcg |
| nt) | gggcggctaacccagccggaggggacgcggcggcg |
| | gccggcgacgaggagcggaaagtggggctggcgcc |
| | cggcgacgtggagcaagtcaccttggcgctcgggg |
| | ccggagccgacaaagacgggaccctgctgctggag |
| | ggcggcggccgcgacgaggggcagcggaggacccc |
| | gcagggcatcgggctcctggccaagaccccgctga |
| | gccgcccagtcaagagaaacaacgccaagtaccgg |
| | cgcatccaaactttgatctacgacgccctggagag |
| | accgcggggctgggcgctgctttaccacgcgttgg |
| | tgttcctgattgtcctggggtgcttgattctggct |
| | gtcctgaccacattcaaggagtatgagactgtctc |
| | gggagactggcttctgttactggagacatttgcta |
| | ttttcatctttggagccgagtttgctttgaggatc |
| | tgggctgctggatgttgctgccgatacaaaggctg |
| | gcggggccgactgaagtttgccaggaagcccctgt |
| | gcatgttggacatctttgtgctgattgcctctgtg |
| | ccagtggttgctgtgggaaaccaaggcaatgttct |
| | ggccacctccctgcgaagcctgcgcttcctgcaga |
| | tcctgcgcatgctgcggatggaccggagaggtggc |
| | acctggaagcttctgggctcagccatctgtgccca |
| | cagcaaagaactcatcacggcctggtacatcggtt |
| | tcctgacactcatcctttcttcatttcttgtctac |
| | ctggttgagaaagacgtcccagaggtggatgcaca |
| | aggagaggagatgaaagaggagtttgagacctatg |
| | cagatgccctgtggtggggcctgatcacactggcc |
| | accattggctatggagacaagacacccaaaacgtg |
| | ggaaggccgtctgattgccgccaccttttccttaa |
| | ttggcgtctccttttttgcccttccagcgggcatc |
| | ctggggtccgggctggccctcaaggtgcaggagca |
| | acaccgtcagaagcactttgagaaaaggaggaagc |
| | cagctgctgagctcattcaggctgcctggaggtat |
| | tatgctaccaaccccaacaggattgacctggtggc |
| | gacatggagattttatgaatcagtcgtctcttttc |
| | ctttcttcaggaaagaacagctggaggcagcatcc |
| | agccaaaagctgggtctcttggatcgggttcgcct |
| | ttctaatcctcgtggtagcaatactaaaggaaagc |
| | tatttacccctctgaatgtagatgccatagaagaa |
| | agtccttctaaagaaccaaagcctgttggcttaaa |
| | caataaagagcgtttccgcacggccttccgcatga |
| | aagcctacgctttctggcagagttctgaagatgcc |
| | gggacaggtgaccccatggcggaagacaggggcta |
| | tgggaatgacttccccatcgaagacatgatcccca |
| | ccctgaaggccgccatccgagccgtcagaattcta |
| | caattccgtctctataaaaaaaaattcaaggagac |
| | tttgaggccttacgatgtgaaggatgtgattgagc |
| | agtattctgccgggcatctcgacatgctttccagg |
| | ataaagtaccttcagacgagaatagatatgatttt |
| | cacccctggacctccctccacgccaaaacacaaga |
| | agtctcagaaagggtcagcattcaccttcccatcc |
| | cagcaatctcccaggaatgaaccatatgtagccag |
| | accatccacatcagaaatcgaagaccaaagcatga |
| | tggggaagtttgtaaaagttgaaagacaggttcag |
| | gacatggggaagaagctggacttcctcgtggatat |
| | gcacatgcaacacatggaacggttgcaggtgcagg |
| | tcacggagtattacccaaccaagggcacctcctcg |
| | ccagctgaagcagagaagaaggaggacaacaggta |
| | ttccgatttgaaaaccatcatctgcaactattctg |
| | agacaggccccccggaaccaccctacagcttccac |
| | caggtgaccattgacaaagtcagcccctatgggtt |
| | ttttgcacatgaccctgtgaacctgccccgagggg |
| | gacccagttctggaaaggttcaggcaactcctcct |
| | tcctcagcaacaacgtatgtggagaggcccacggt |
| | cctgcctatcttgactcttctcgactcccgagtga |
| | gctgccactcccaggctgacctgcagggcccctac |
| | tcggaccgaatctccccccggcagagacgtagcat |
| | cacgcgagacagtgacacacctctgtccctgatgt |
| | cggtcaaccacgaggagctggagaggtctccaagt |
| | ggcttcagcatctcccaggacagagatgattatgt |
| | gttcggccccaatggggggtcgagctggatgaggg |
| | agaagcggtacctcgccgagggtgagacggacaca |
| | gacacggaccccttcacgcccagcggctccatgcc |
| | tctgtcgtccacaggggat |
| 2 | gggatttctgattcagtatggaccccttccaataa |
| (KCNQ3 | gcccatttaaMGLKARRAAGAAGGGGDGGGGGGGA |
| AA) | ANPAGGDAAAAGDEERKVGLAPGDVEQVTLALGAG |
| | ADKDGTLLLEGGGRDEGQRRTPQGIGLLAKTPLSR |
| | PVKRNNAKYRRIQTLIYDALERPRGWALLYHALVF |
| | LIVLGCLILAVLTTFKEYETVSGDWLLLLETFAI |
| | FIFGAEFALRIWAAGCCCRYKGWRGRLKFARKPLC |
| | MLDIFVLIASVPVVAVGNQGNVLATSLRSLRFLQI |
| | LRMLRMDRRGGTWKLLGSAICAHSKELITAWYIGF |
| | LTLILSSFLVYLVEKDVPEVDAQGEEMKEEFETYA |
| | DALWWGLITLATIGYGDKTPKTWEGRLIAATFSLI |
| | GVSFFALPAGILGSGLALKVQEQHRQKHFEKRRKP |
| | AAELIQAAWRYYATNPNRIDLVATWRFYESVVSFP |
| | FFRKEQLEAASSQKLGLLDRVRLSNPRGSNTKGKL |
| | FTPLNVDAIEESPSKEPKPVGLNNKERFRTAFRMK |
| | AYAFWQSSEDAGTGDPMAEDRGYGNDFPIEDMIPT |
| | LKAAIRAVRILQFRLYKKKFKETLRPYDVKDVIEQ |
| | YSAGHLDMLSRIKYLQTRIDMIFTPGPPSTPKHKK |
| | SQKGSAFTFPSQQSPRNEPYVARPSTSEIEDQSMM |
| | GKFVKVERQVQDMGKKLDFLVDMHMQHMERLQVQV |
| | TEYYPTKGTSSPAEAEKKEDNRYSDLKTIICNYSE |
| | TGPPEPPYSFHQVTIDKVSPYGFFAHDPVNLPRGG |
| | PSSGKVQATPPSSATTYVERPTVLPILTLLDSRVS |
| | CHSQADLQGPYSDRISPRQRRSITRDSDTPLSLMS |
| | VNHEELERSPSGFSISQDRDDYVFGPNGGSSWMRE |
| | KRYLAEGETDTDTDPFTPSGSMPLSSTGDGISDSV |
| | WTPSNKPI |

In some aspects, the products and methods are designed to treat KCNQ3-related disorders resulting from mutations in the KCNQ3 gene. Such KCNQ3-related disorders include, but are not limited to, DEE. The products and methods are designed to treat or reduce or inhibit the mutant or pathogenic expression of KCNQ3 resulting from various mutations in the KCNQ3 gene. In some aspects, such mutations in the KCNQ3 gene include, but are not limited to, R230C, R230H, R230S, and R227Q. Each of these particular mutations have been reported to be a gain-of-function mutation. The disclosure includes products and methods for treating KCNQ3-related disorders resulting from such various KCNQ3 gene mutations.

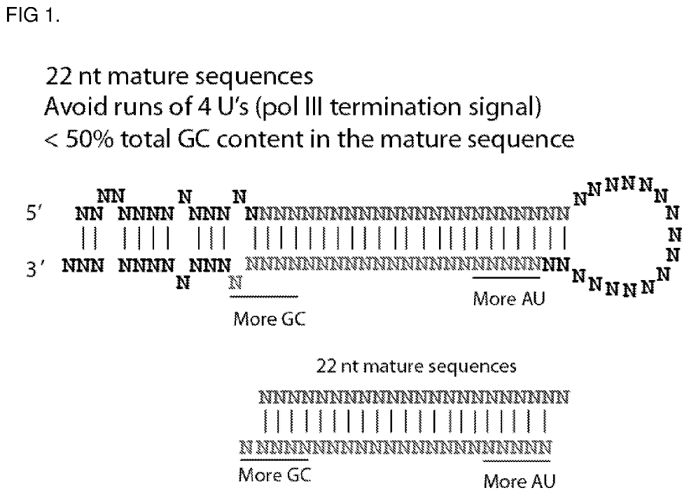

The disclosure provides nucleic acids encoding microRNA (miRNA) targeting KCNQ3 and variants of KCNQ3, and reducing or inhibiting the expression of KCNQ3 and variants of KCNQ3. The nucleic acids comprise nucleotide sequences encoding microRNA (miRNA) targeting KCNQ3 and variants of KCNQ3. The miRNA nucleotide sequences were specifically designed and selected with the use of an algorithm, which was developed to predict effective artificial microRNAs (FIG. 1 shows criteria for selection). Using human KCNQ3 cDNA as query sequence, the algorithm identified 152 prospective microRNAs that fit the criteria listed in FIG. 1. A second layer of selection was added by incorporating species conservation. Specifically, mouse, rat, and human KCNQ3 cDNAs were aligned. Of the 152 prospective microRNAs, only 15 miRNAs contained perfect 22 nucleotide base-pairing between the antisense guide strand and the KCNQ3 target sites of the three species (i.e., mouse, rat, and human). Seven of the 15 miRNAs were selected for construction and empirical testing, as described herein.

The disclosure provides nucleic acids encoding miRNA targeting KCNQ3 and variants of KCNQ3, wherein the nucleic acids also comprise promoter nucleotide sequences.

The disclosure provides nucleic acids comprising the RNA sequence targeted by the miRNA.

The disclosure provides KCNQ3 sequences that the miRNA sequences are designed to target.

The disclosure includes various nucleic acids comprising, consisting essentially of, or consisting of the various nucleotide sequences described herein. In some aspects, the nucleic acid comprises the nucleotide sequence. In some aspects, the nucleic acid consists essentially of the nucleotide sequence. In some aspects, the nucleic acid consists of the nucleotide sequence.

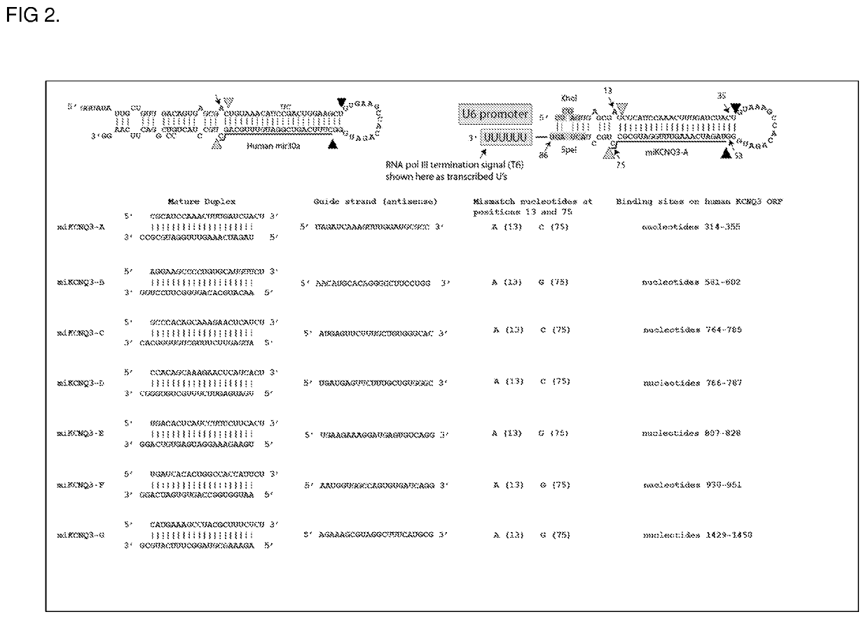

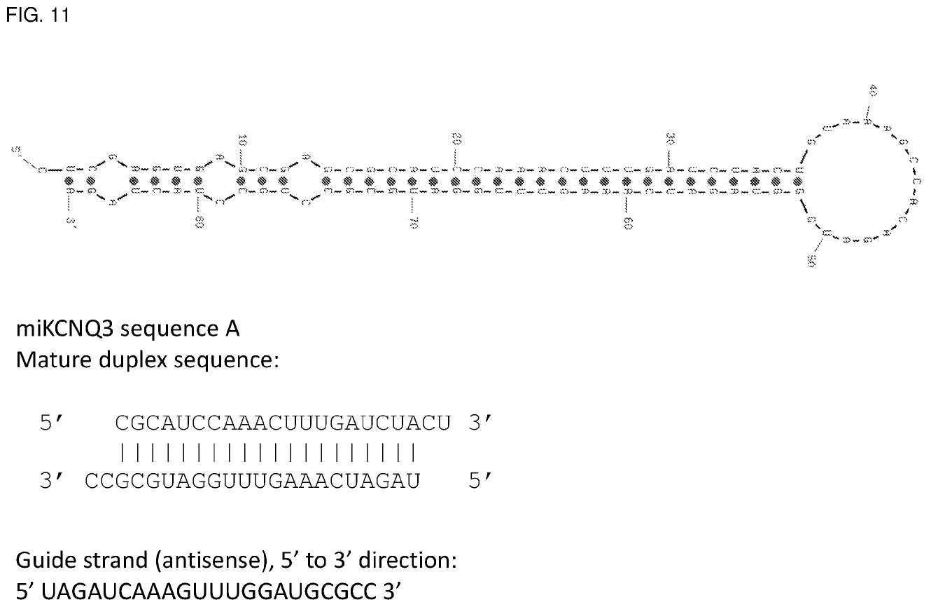

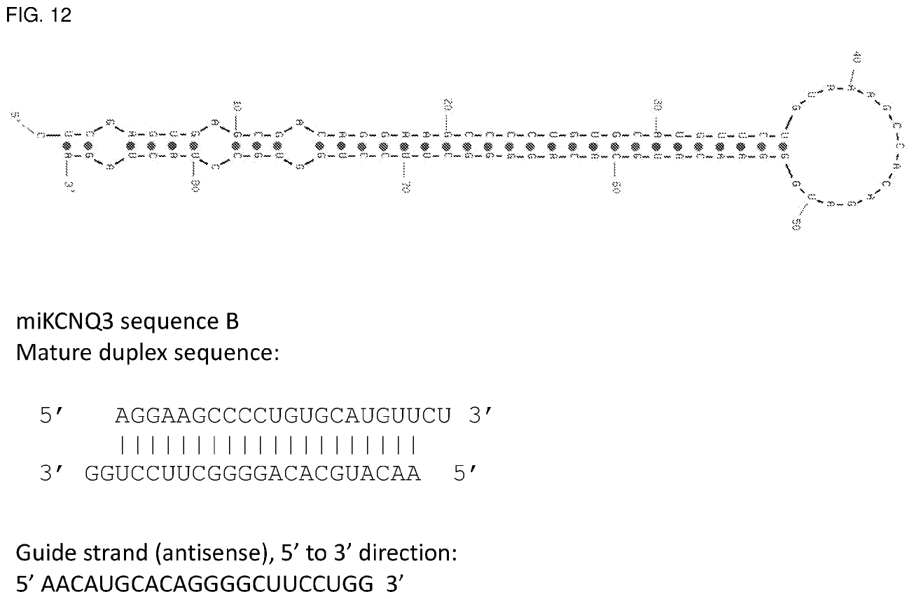

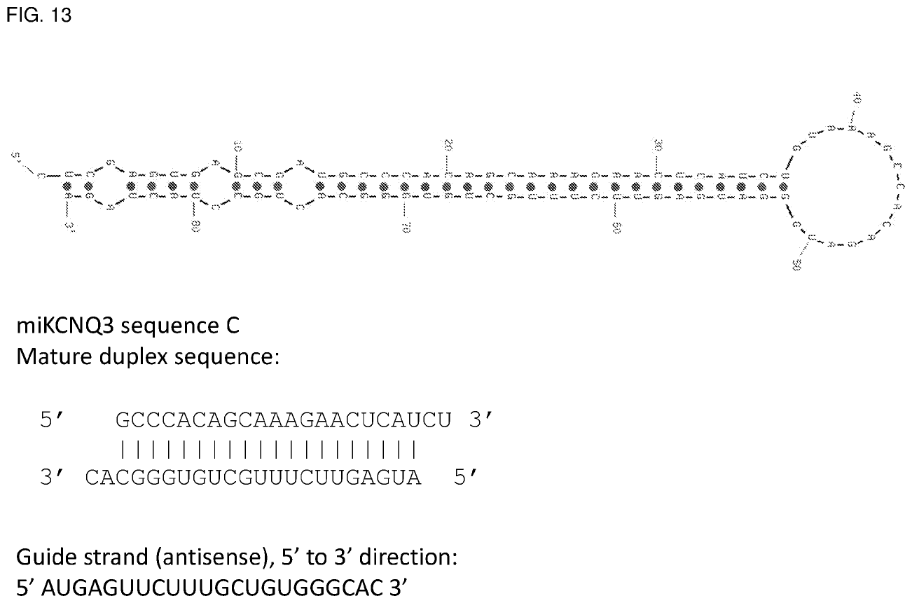

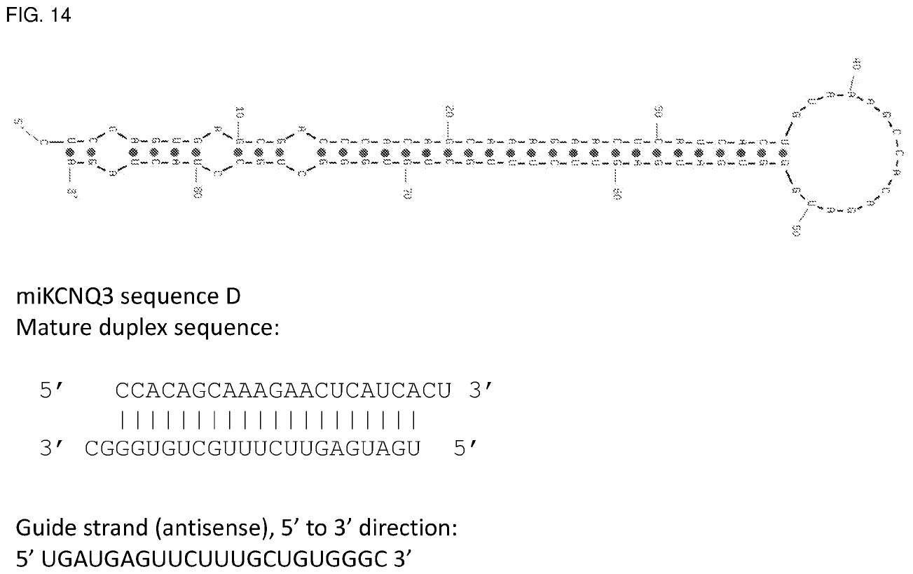

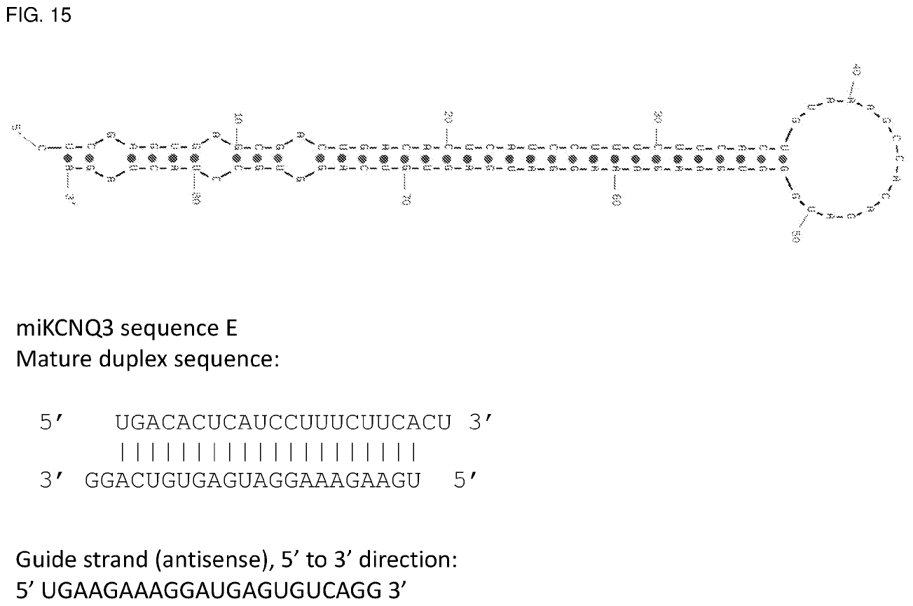

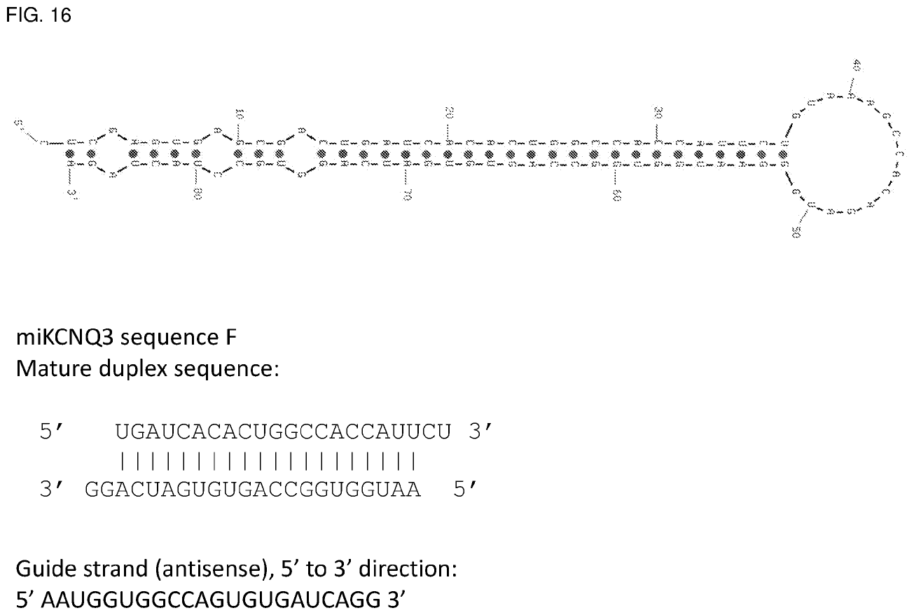

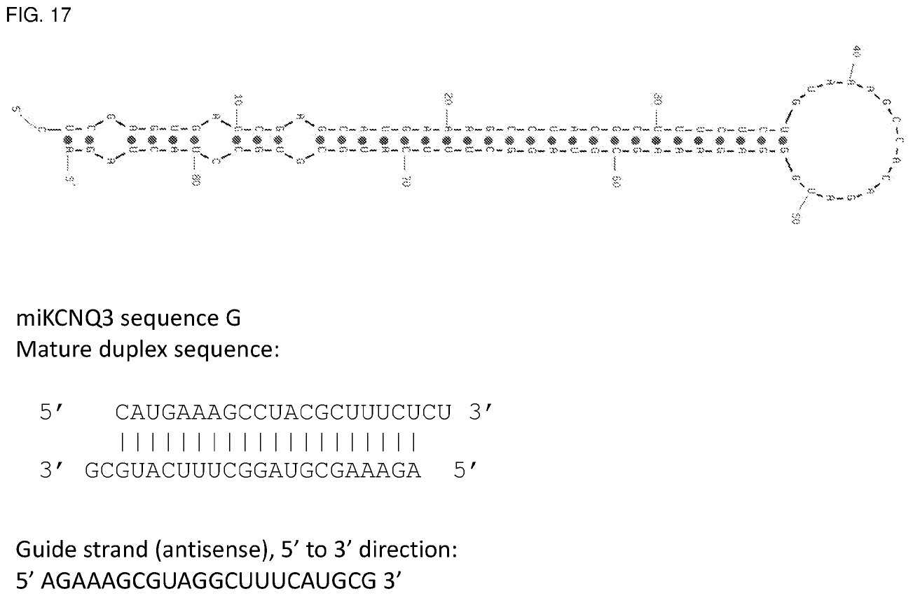

Exemplary nucleotide sequences used in miRNA targeting of KCNQ3 described herein include, but are not limited to, those identified in Table 2 below and in FIGS. 2 and 11-17.

TABLE 2
Nucleic acids of the disclosure

| | | | | | | | RNA | | |
|---|---|---|---|---|---|---|---|---|---|
| | | | | | | | sequence | | |
| | | | | | | | (Guide | | |
| | | | | | | | strand | | |
| | | | | | | | (antisense | | |
| | | SEQ | | SEQ | DNA sequence | SEQ | sequence, | SEQ | KCNQ3 target |
| miRNA | miRNA | ID | DNA sequence | ID | with the | ID | 5′ to 3′ | ID | RNA |
| # | name | NO: | encoding miRNA | NO: | U6 promoter | NO: | direction)) | NO: | sequence |
| 1 | miKCNQ3 | 3 | TAGATCAAAGTTTG | 10 | GACGCCGCCATCTCTAGGC | 17 | UAGAUCAAA | 24 | GGCGCAUCCAAA |
| | A | | GATGCGCC | | CCGCGCCGGCCCCCTCGC | | GUUUGGAUG | | CUUUGAUCUA |
| | | | DNA sequence | | ACAGACTTGTGGGAGAAGC | | CGCC | | |
| | | | encoding miRNA | | TCGGCTACTCCCCTGCCCC | | | | |
| | | | including 5′ XhoI | | GGTTAATTTGCATATAATAT | | | | |
| | | | (CTCGAG) and 3′ | | TTCCTAGTAACTATAGAGGC | | | | |
| | | | hybrid Xba/SpeI | | TTAATGTGCGATAAAAGACA | | | | |
| | | | (ACTAGA) | | GATAATCTGTTCTTTTTAATA | | | | |
| | | | restriction sites | | CTAGCTACATTTTACATGAT | | | | |
| | | 31 | CTCGAGTGAGCGA | | AGGCTTGGATTTCTATAAGA | | | | |
| | | | GCGCATCCAAACTT | | GATACAAATACTAAATTATT | | | | |
| | | | TGATCTACTGTAAA | | ATTTTAAAAAACAGCACAAA | | | | |
| | | | GCCACAGATGGGTA | | AGGAAACTCACCCTAACTGT | | | | |
| | | | GATCAAAGTTTGGA | | AAAGTAATTGTGTGTTTTGA | | | | |
| | | | TGCGCCTGCCTACT | | GACTATAAATATCCCTTGGA | | | | |
| | | | AGA | | GAAAAGCCTTGTTTGCGTTT | | | | |
| | | | | | AGTGAACCGTCAGATGGTA | | | | |
| | | | | | CCGTTTAAACTCGAGTGAG | | | | |
| | | | | | CGAGCGCATCCAAACTTTG | | | | |
| | | | | | ATCTACTGTAAAGCCACAGA | | | | |
| | | | | | TGGGTAGATCAAAGTTTGG | | | | |
| | | | | | ATGCGCCTGCCTACTAGAG | | | | |
| | | | | | CGGCCGCCACAGCGGGGA | | | | |
| | | | | | GATCCAGACATGATAAGATA | | | | |
| | | | | | CATTTTTT | | | | |
| 2 | miKCNQ3 | 4 | AACATGCACAGGGG | 11 | GACGCCGCCATCTCTAGGC | 18 | AACAUGCAC | 25 | CCAGGAAGCCC |
| | B | | CTTCCTGG | | CCGCGCCGGCCCCCTCGC | | AGGGGCUUC | | CUGUGCAUGUU |
| | | | DNA sequence | | ACAGACTTGTGGGAGAAGC | | CUGG | | |
| | | | encoding miRNA | | TCGGCTACTCCCCTGCCCC | | | | |
| | | | including 5′ XhoI | | GGTTAATTTGCATATAATAT | | | | |
| | | | (CTCGAG) and 3′ | | TTCCTAGTAACTATAGAGGC | | | | |
| | | | hybrid Xba/SpeI | | TTAATGTGCGATAAAAGACA | | | | |
| | | | (ACTAGA) | | GATAATCTGTTCTTTTTAATA | | | | |
| | | | restriction sites | | CTAGCTACATTTTACATGAT | | | | |
| | | 32 | CTCGAGTGAGCGAC | | AGGCTTGGATTTCTATAAGA | | | | |
| | | | AGGAAGCCCCTGTG | | GATACAAATACTAAATTATT | | | | |
| | | | CATGTTCTGTAAAG | | ATTTTAAAAAACAGCACAAA | | | | |
| | | | CCACAGATGGGAAC | | AGGAAACTCACCCTAACTGT | | | | |
| | | | ATGCACAGGGGCTT | | AAAGTAATTGTGTGTTTTGA | | | | |
| | | | CCTGGTGCCTACTA | | GACTATAAATATCCCTTGGA | | | | |
| | | | GA | | GAAAAGCCTTGTTTGCGTTT | | | | |
| | | | | | AGTGAACCGTCAGATGGTA | | | | |
| | | | | | CCGTTTAAACTCGAGTGAG | | | | |
| | | | | | CGACAGGAAGCCCCTGTGC | | | | |
| | | | | | ATGTTCTGTAAAGCCACAGA | | | | |
| | | | | | TGGGAACATGCACAGGGGC | | | | |
| | | | | | TTCCTGGTGCCTACTAGAG | | | | |
| | | | | | CGGCCGCCACAGCGGGGA | | | | |
| | | | | | GATCCAGACATGATAAGATA | | | | |
| | | | | | CATTTTTT | | | | |
| 3 | miKCNQ3 | 5 | ATGAGTTCTTTGCT | 12 | GACGCCGCCATCTCTAGGC | 19 | AUGAGUUCU | 26 | GUGCCCACAGC |
| | C | | GTGGGCAC | | CCGCGCCGGCCCCCTCGC | | UUGCUGUGG | | AAAGAACUCAU |
| | | | DNA sequence | | ACAGACTTGTGGGAGAAGC | | GCAC | | |
| | | | encoding miRNA | | TCGGCTACTCCCCTGCCCC | | | | |
| | | | including 5′ XhoI | | GGTTAATTTGCATATAATAT | | | | |
| | | | (CTCGAG) and 3′ | | TTCCTAGTAACTATAGAGGC | | | | |
| | | | hybrid Xba/SpeI | | TTAATGTGCGATAAAAGACA | | | | |
| | | | (ACTAGA) | | GATAATCTGTTCTTTTTAATA | | | | |
| | | | restriction sites | | CTAGCTACATTTTACATGAT | | | | |
| | | 33 | CTCGAGTGAGCGAT | | AGGCTTGGATTTCTATAAGA | | | | |
| | | | GCCCACAGCAAAGA | | GATACAAATACTAAATTATT | | | | |
| | | | ACTCATCTGTAAAG | | ATTTTAAAAAACAGCACAAA | | | | |
| | | | CCACAGATGGGATG | | AGGAAACTCACCCTAACTGT | | | | |
| | | | AGTTCTTTGCTGTG | | AAAGTAATTGTGTGTTTTGA | | | | |
| | | | GGCACTGCCTACTA | | GACTATAAATATCCCTTGGA | | | | |
| | | | GA | | GAAAAGCCTTGTTTGCGTTT | | | | |
| | | | | | AGTGAACCGTCAGATGGTA | | | | |
| | | | | | CCGTTTAAACTCGAGTGAG | | | | |
| | | | | | CGATGCCCACAGCAAAGAA | | | | |
| | | | | | CTCATCTGTAAAGCCACAGA | | | | |
| | | | | | TGGGATGAGTTCTTTGCTGT | | | | |
| | | | | | GGGCACTGCCTACTAGAGC | | | | |
| | | | | | GGCCGCCACAGCGGGGAG | | | | |
| | | | | | ATCCAGACATGATAAGATAC | | | | |
| | | | | | ATTTTTT | | | | |
| 4 | miKCNQ3 | 6 | TGATGAGTTCTTTG | 13 | GACGCCGCCATCTCTAGGC | 20 | UGAUGAGUU | 27 | GCCCACAGCAA |
| | D | | CTGTGGGC | | CCGCGCCGGCCCCCTCGC | | CUUUGCUGU | | AGAACUCAUCA |
| | | | DNA sequence | | ACAGACTTGTGGGAGAAGC | | GGGC | | |
| | | | encoding miRNA | | TCGGCTACTCCCCTGCCCC | | | | |
| | | | including 5′ XhoI | | GGTTAATTTGCATATAATAT | | | | |
| | | | (CTCGAG) and 3′ | | TTCCTAGTAACTATAGAGGC | | | | |
| | | | hybrid Xba/SpeI | | TTAATGTGCGATAAAAGACA | | | | |
| | | | (ACTAGA) | | GATAATCTGTTCTTTTTAATA | | | | |
| | | | restriction sites | | CTAGCTACATTTTACATGAT | | | | |
| | | 34 | CTCGAGTGAGCGAC | | AGGCTTGGATTTCTATAAGA | | | | |
| | | | CCACAGCAAAGAAC | | GATACAAATACTAAATTATT | | | | |
| | | | TCATCACTGTAAAG | | ATTTTAAAAAACAGCACAAA | | | | |
| | | | CCACAGATGGGTGA | | AGGAAACTCACCCTAACTGT | | | | |
| | | | TGAGTTCTTTGCTG | | AAAGTAATTGTGTGTTTTGA | | | | |
| | | | TGGGCTGCCTACTA | | GACTATAAATATCCCTTGGA | | | | |
| | | | GA | | GAAAAGCCTTGTTTGCGTTT | | | | |
| | | | | | AGTGAACCGTCAGATGGTA | | | | |
| | | | | | CCGTTTAAACTCGAGTGAG | | | | |
| | | | | | CGACCCACAGCAAAGAACT | | | | |
| | | | | | CATCACTGTAAAGCCACAG | | | | |
| | | | | | ATGGGTGATGAGTTCTTTGC | | | | |
| | | | | | TGTGGGCTGCCTACTAGAG | | | | |
| | | | | | CGGCCGCCACAGCGGGGA | | | | |
| | | | | | GATCCAGACATGATAAGATA | | | | |
| | | | | | CATTTTTT | | | | |
| 5 | miKCNQ3 | 7 | TGAAGAAAGGATGA | 14 | GACGCCGCCATCTCTAGGC | 21 | UGAAGAAAG | 28 | CCUGACACUCA |
| | E | | GTGTCAGG | | CCGCGCCGGCCCCCTCGC | | GAUGAGUGU | | UCCUUUCUUCA |
| | | 35 | DNA sequence | | ACAGACTTGTGGGAGAAGC | | CAGG | | |
| | | | encoding miRNA | | TCGGCTACTCCCCTGCCCC | | | | |
| | | | including 5′ XhoI | | GGTTAATTTGCATATAATAT | | | | |
| | | | (CTCGAG) and 3′ | | TTCCTAGTAACTATAGAGGC | | | | |
| | | | hybrid Xba/SpeI | | TTAATGTGCGATAAAAGACA | | | | |
| | | | (ACTAGA) | | GATAATCTGTTCTTTTTAATA | | | | |
| | | | restriction sites | | CTAGCTACATTTTACATGAT | | | | |
| | | | CTCGAGTGAGCGAC | | AGGCTTGGATTTCTATAAGA | | | | |
| | | | TGACACTCATCCTT | | GATACAAATACTAAATTATT | | | | |
| | | | TCTTCACTGTAAAG | | ATTTTAAAAAACAGCACAAA | | | | |
| | | | CCACAGATGGGTGA | | AGGAAACTCACCCTAACTGT | | | | |
| | | | AGAAAGGATGAGTG | | AAAGTAATTGTGTGTTTTGA | | | | |
| | | | TCAGGTGCCTACTA | | GACTATAAATATCCCTTGGA | | | | |
| | | | GA | | GAAAAGCCTTGTTTGCGTTT | | | | |
| | | | | | AGTGAACCGTCAGATGGTA | | | | |
| | | | | | CCGTTTAAACTCGAGTGAG | | | | |
| | | | | | CGACTGACACTCATCCTTTC | | | | |
| | | | | | TTCACTGTAAAGCCACAGAT | | | | |
| | | | | | GGGTGAAGAAAGGATGAGT | | | | |
| | | | | | GTCAGGTGCCTACTAGAGC | | | | |
| | | | | | GGCCGCCACAGCGGGGAG | | | | |
| | | | | | ATCCAGACATGATAAGATAC | | | | |
| | | | | | ATTTTTT | | | | |
| 6 | miKCNQ3 | 8 | AATGGTGGCCAGTG | 15 | GACGCCGCCATCTCTAGGC | 22 | AAUGGUGGC | 29 | CCUGAUCACAC |
| | F | | TGATCAGG | | CCGCGCCGGCCCCCTCGC | | CAGUGUGAU | | UGGCCACCAUU |
| | | 36 | DNA sequence | | ACAGACTTGTGGGAGAAGC | | CAGG | | |
| | | | encoding miRNA | | TCGGCTACTCCCCTGCCCC | | | | |
| | | | including 5′ XhoI | | GGTTAATTTGCATATAATAT | | | | |
| | | | (CTCGAG) and 3′ | | TTCCTAGTAACTATAGAGGC | | | | |
| | | | hybrid Xba/SpeI | | TTAATGTGCGATAAAAGACA | | | | |
| | | | (ACTAGA) | | GATAATCTGTTCTTTTTAATA | | | | |
| | | | restriction sites | | CTAGCTACATTTTACATGAT | | | | |
| | | | CTCGAGTGAGCGAC | | AGGCTTGGATTTCTATAAGA | | | | |
| | | | TGATCACACTGGCC | | GATACAAATACTAAATTATT | | | | |
| | | | ACCATTCTGTAAAG | | ATTTTAAAAAACAGCACAAA | | | | |
| | | | CCACAGATGGGAAT | | AGGAAACTCACCCTAACTGT | | | | |
| | | | GGTGGCCAGTGTG | | AAAGTAATTGTGTGTTTTGA | | | | |
| | | | ATCAGGTGCCTACT | | GACTATAAATATCCCTTGGA | | | | |
| | | | AGA | | GAAAAGCCTTGTTTGCGTTT | | | | |
| | | | | | AGTGAACCGTCAGATGGTA | | | | |
| | | | | | CCGTTTAAACTCGAGTGAG | | | | |
| | | | | | CGACTGATCACACTGGCCA | | | | |
| | | | | | CCATTCTGTAAAGCCACAGA | | | | |
| | | | | | TGGGAATGGTGGCCAGTGT | | | | |
| | | | | | GATCAGGTGCCTACTAGAG | | | | |
| | | | | | CGGCCGCCACAGCGGGGA | | | | |
| | | | | | GATCCAGACATGATAAGATA | | | | |
| | | | | | CATTTTTT | | | | |
| 7 | miKCNQ3 | 9 | AGAAAGCGTAGGCT | 16 | GACGCCGCCATCTCTAGGC | 23 | AGAAAGCGU | 30 | CGCAUGAAAGC |
| | G | | TTCATGCG | | CCGCGCCGGCCCCCTCGC | | AGGCUUUCA | | CUACGCUUUCU |
| | | 37 | DNA sequence | | ACAGACTTGTGGGAGAAGC | | UGCG | | |
| | | | encoding miRNA | | TCGGCTACTCCCCTGCCCC | | | | |
| | | | including 5′ XhoI | | GGTTAATTTGCATATAATAT | | | | |
| | | | (CTCGAG) and 3′ | | TTCCTAGTAACTATAGAGGC | | | | |
| | | | hybrid Xba/SpeI | | TTAATGTGCGATAAAAGACA | | | | |
| | | | (ACTAGA) | | GATAATCTGTTCTTTTTAATA | | | | |
| | | | restriction sites | | CTAGCTACATTTTACATGAT | | | | |
| | | | CTCGAGTGAGCGA | | AGGCTTGGATTTCTATAAGA | | | | |
| | | | GCATGAAAGCCTAC | | GATACAAATACTAAATTATT | | | | |
| | | | GCTTTCTCTGTAAA | | ATTTTAAAAAACAGCACAAA | | | | |
| | | | GCCACAGATGGGA | | AGGAAACTCACCCTAACTGT | | | | |
| | | | GAAAGCGTAGGCTT | | AAAGTAATTGTGTGTTTTGA | | | | |
| | | | TCATGCGTGCCTAC | | GACTATAAATATCCCTTGGA | | | | |
| | | | TAGA | | GAAAAGCCTTGTTTGCGTTT | | | | |
| | | | | | AGTGAACCGTCAGATGGTA | | | | |
| | | | | | CCGTTTAAACTCGAGTGAG | | | | |
| | | | | | CGAGCATGAAAGCCTACGC | | | | |
| | | | | | TTTCTCTGTAAAGCCACAGA | | | | |
| | | | | | TGGGAGAAAGCGTAGGCTT | | | | |
| | | | | | TCATGCGTGCCTACTAGAG | | | | |
| | | | | | CGGCCGCCACAGCGGGGA | | | | |
| | | | | | GATCCAGACATGATAAGATA | | | | |
| | | | | | CATTTTTT | | | | |

Exemplary nucleotide sequences are set out in Table 2 above and in FIGS. 2 and 11-17. In some instances, the miRNA has one binding site on KCNQ3.

In some aspects, a nucleic acid of the disclosure comprises a nucleotide sequence comprising at least or about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence set forth in any one of SEQ ID NOs: 3-37. See Table 2.

In some aspects, a nucleic acid of the disclosure comprises a nucleotide sequence comprising at least 90% identity to the sequence set forth in any one of SEQ ID NOs: 3-9; the nucleotide sequence set forth in any one of SEQ ID NOs: 3-9; a nucleotide sequence comprising at least 90% identity to the sequence set forth in any one of SEQ ID NOs: 10-16; the nucleotide sequence set forth in any one of SEQ ID NOs: 10-16; a nucleotide sequence that encodes the RNA sequence set forth in any one of SEQ ID NOs: 17-23; or a nucleotide sequence that specifically hybridizes to the KCNQ3 sequence set forth in any one of SEQ ID NOs: 24-30.

In some aspects, a nucleic acid of the disclosure comprises a nucleotide sequence comprising at least 90% identity to the sequence set forth in any one of SEQ ID NOs: 31-37, or the nucleotide sequence set forth in any one of SEQ ID NOs: 31-37. As set out in FIG. 2, SEQ ID NOs: 31-37 are the DNA sequences encoding miRNAs including 5′ XhoI (CTCGAG) and 3′ hybrid Xba/SpeI (ACTAGA) restriction sites. These DNA sequences comprise the underlined 22-base nucleotide sequences (SEQ ID NOs: 3-9) which encode the 22-nucleotide miRNA sequences (SEQ ID NOs: 17-23).

In some aspects, a nucleic acid of the disclosure comprises or consists of a nucleotide sequence set forth in any one of SEQ ID NOs: 17-23, or a nucleotide sequence set forth in any one of SEQ ID NOs: 24-30.

In some aspects, the disclosure includes the use of RNA interference to reduce or inhibit KCNQ3 expression. RNA interference (RNAi) is a mechanism of gene regulation in eukaryotic cells that has been considered for the treatment of various diseases. RNAi refers to post-transcriptional control of gene expression mediated by miRNAs. The miRNAs are small (about 21-25 nucleotides), noncoding RNAs that share sequence homology and base-pair with sequence target sites of cognate messenger RNAs (mRNAs). The interaction between the miRNAs and mRNAs directs cellular gene silencing machinery inducing mRNA decay and/or preventing mRNA translation into protein.

As an understanding of natural RNAi pathways has developed, researchers have designed artificial shRNAs and snRNAs for use in regulating expression of target genes for treating disease. Several classes of small RNAs are known to trigger RNAi processes in mammalian cells, including short (or small) interfering RNA (siRNA), and short (or small) hairpin RNA (shRNA) and microRNA (miRNA), which constitute a similar class of vector-expressed triggers [Davidson et al., Nat. Rev. Genet. 12:329-40, 2011; Harper, Arch. Neurol. 66:933-8, 2009]. shRNA and miRNA are expressed in vivo from plasmid- or virus-based vectors and may thus achieve long term gene silencing with a single administration, for as long as the vector is present within target cell nuclei and the driving promoter is active (Davidson et al., Methods Enzymol. 392:145-73, 2005). Importantly, this vector-expressed approach leverages the decades-long advancements already made in the gene therapy field, but instead of expressing protein coding genes, the vector cargo in RNAi therapy strategies are artificial shRNA or miRNA cassettes targeting disease genes-of-interest.

In some embodiments, the products and methods of the disclosure comprise microRNA (miRNA). MicroRNAs (miRNAs) are a class of non-coding RNAs that play important roles in RNA silencing and in regulating gene expression. The majority of miRNAs are transcribed from DNA sequences into primary miRNAs and processed into precursor miRNAs, and finally mature miRNAs. In most cases, miRNAs interact with the 3′ untranslated region (3′ UTR) of target mRNAs to induce mRNA degradation and translational repression. However, interaction of miRNAs with other regions, including the 5′ UTR, coding sequence, and gene promoters, have also been reported. Under certain conditions, miRNAs can also activate translation or regulate transcription. The interaction of miRNAs with their target genes is dynamic and dependent on many factors, such as subcellular location of miRNAs, the abundancy of miRNAs and target mRNAs, and the affinity of miRNA-mRNA interactions.

Most studies to date have shown that miRNAs bind to a specific sequence at the 3′ UTR of their target mRNAs to induce translational repression and mRNA deadenylation and decapping. miRNA binding sites have also been detected in other mRNA regions including the 5′ UTR and coding sequence, as well as within promoter regions. The binding of miRNAs to 5′ UTR and coding regions have silencing effects on gene expression while miRNA interaction with promoter region has been reported to induce transcription.

In various aspects, polymerase II promoters and polymerase III promoters, such as U6 and H1, are used. In some aspects, U6 miRNAs are used. In some aspects, H1 miRNAs are used. Thus, in some aspects, U6 miRNA or H1 miRNA are used to further reduce, inhibit, knockdown, or interfere with KCNQ3 gene expression. Traditional small/short hairpin RNA (shRNA) sequences are usually transcribed inside the cell nucleus from a vector containing a Pol III promoter, such as U6. The endogenous U6 promoter normally controls expression of the U6 RNA, a small nuclear RNA (snRNA) involved in splicing, and has been well-characterized [Kunkel et al., Nature. 322(6074):73-7 (1986); Kunkel et al., Genes Dev. 2(2):196-204 (1988); Paule et al., Nucleic Acids Res. 28(6):1283-98 (2000)]. In some aspects, the U6 or H1 promoter is used to control vector-based expression of shRNA molecules in mammalian cells [Paddison et al., Proc. Natl. Acad. Sci. USA 99(3):1443-8 (2002); Paul et al., Nat. Biotechnol. 20(5):505-8 (2002); Medina et al., Curr. Opin. Mol. Ther. 1:580-94 (1999)] because (1) the promoter is recognized by RNA polymerase III (poly Ill) and controls high-level, constitutive expression of shRNA; (2) the Pol III promoter possesses greater capacity than RNA polymerase I to synthesize shRNA of high yield [Boden et al., Nucleic Acids Res. 32:1154-8 (2004); Xia et al., Neurodegenerative Dis. 2:220-31 (2005)]; (3) the Pol III promoters are consistent of compact sequence and simple terminator that are easy to handle [Medina et al. (1999) supra]; and (2) the promoter is active in most mammalian cell types. In some aspects, the promoter is a type III Pol Ill promoter in that all elements required to control expression of the shRNA are located upstream of the transcription start site [Paule et al., Nucleic Acids Res. 28(6):1283-98 (2000)]. The disclosure includes both murine and human U6 promoters. The shRNA containing the sense and antisense sequences from a target gene connected by a loop is transported from the nucleus into the cytoplasm where Dicer processes it into small/short interfering RNAs (siRNAs).

In various aspects, the miRNA is expressed under various promoters and/or enhancers including, but not limited to, a U6 promoter, a U7 promoter, an H1 promoter, a T7 promoter, a tRNA promoter, an EF1-alpha promoter, a minimal EF1-alpha promoter, an unc45b promoter, a CK1 promoter, a CK6 promoter, a CK7 promoter, a CK8 promoter, a miniCMV promoter, a CMV enhancer and/or promoter, a ubiquitous promoter, a neuronal-specific or brain-specific promoter. In some aspects, such neuronal-specific or brain-specific promoter is human Synapsin1 (hSyn1), neuron-specific enolase (Nse), MeCP2, mDLX, mDLX5/6, calmodulin-dependent kinase II (CaMKII or Camk2a). In some aspects, the promoter and/or enhancer is any of the promoters and/or enhancers disclosed by Haery et al. (Front Neuroanat. 2019; 13:93; PMID: 31849618, incorporated herein by reference in its entirety) including, but not limited to, the promoters and enhancers disclosed in Tables 3 and 4 of Haery et al.

In some embodiments, the disclosure includes a vector comprising any of the nucleic acids described herein. Thus, embodiments of the disclosure utilize vectors (for example, viral vectors, such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle, or small molecule) to deliver the nucleic acids disclosed herein.

In some embodiments, the vectors are AAV vectors. In some aspects, the vectors are single stranded AAV vectors. In some aspects the AAV is recombinant AAV (rAAV). In some aspects, the rAAV lack rep and cap genes. In some aspects, rAAV are self-complementary (sc)AAV.

In some aspects, the viral vector is an adeno-associated virus (AAV), such as an AAV1, AAV2, AAV3, AAV4, AAV5, AAV6, AAV7, AAV8, AAV9, AAV.rh74, AAV.rh8, AAV.rh10, AAV11, AAV12, AAV13, AAV-anc80, AAV-B1, AAV.PHP.EB, or AAVv66.

In some aspects, the viral vector is an adeno-associated virus (AAV), such as an AAV1 (i.e., an AAV containing AAV1 inverted terminal repeats (ITRs) and/or AAV1 capsid proteins), AAV2 (i.e., an AAV containing AAV2 ITRs and/or AAV2 capsid proteins), AAV3 (i.e., an AAV containing AAV3 ITRs and/or AAV3 capsid proteins), AAV4 (i.e., an AAV containing AAV4 ITRs and/or AAV4 capsid proteins), AAV5 (i.e., an AAV containing AAV5 ITRs and/or AAV5 capsid proteins), AAV6 (i.e., an AAV containing AAV6 ITRs and/or AAV6 capsid proteins), AAV7 (i.e., an AAV containing AAV7 ITRs and/or AAV7 capsid proteins), AAV8 (i.e., an AAV containing AAV8 ITRs and/or AAV8 capsid proteins), AAV9 (i.e., an AAV containing AAV9 ITRs and/or AAV9 capsid proteins), AAV.rh74 (i.e., an AAV containing AAV.rh74 ITRs and/or AAV.rh74 capsid proteins), AAV.rh8 (i.e., an AAV containing AAV.rh8 ITRs and/or AAV.rh8 capsid proteins), AAV.rh10 (i.e., an AAV containing AAV.rh10 ITRs and/or AAV.rh10 capsid proteins), AAV11 (i.e., an AAV containing AAV11 ITRs and/or AAV11 capsid proteins), AAV12 (i.e., an AAV containing AAV12 ITRs and/or AAV12 capsid proteins), AAV13 (i.e., an AAV containing AAV13 ITRs and/or AAV13 capsid proteins), AAV-anc80 (i.e., an AAV containing AAV-anc80 ITRs and/or AAV-anc80 capsid proteins), AAV-B1 (i.e., an AAV containing AAV-B1 ITRs and/or AAV-B1 capsid proteins), AAV.PHP.EB (i.e., an AAV containing AAV-PHP.EB ITRs and/or AAV-PHP.EB capsid proteins), or AAVv66 (i.e., an AAV containing AAVv66 ITRs and/or AAVv66 capsid proteins).

In some aspects, the disclosure utilizes adeno-associated virus (AAV) to deliver nucleic acids encoding the miRNA. AAV is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the complete genome of AAV1 is provided in GenBank Accession No. NC_002077; the complete genome of AAV2 is provided in GenBank Accession No. NC_001401 and Srivastava et al., J. Virol., 45: 555-564 {1983); the complete genome of AAV3 is provided in GenBank Accession No. NC_1829; the complete genome of AAV4 is provided in GenBank Accession No. NC_001829; the AAV5 genome is provided in GenBank Accession No. AF085716; the complete genome of AAV6 is provided in GenBank Accession No. NC_00 1862; at least portions of AAV7 and AAV8 genomes are provided in GenBank Accession Nos. AX753246 and AX753249, respectively (see also U.S. Pat. Nos. 7,282,199 and 7,790,449 relating to AAV8); the AAV9 genome is provided in Gao et al., J. Virol., 78: 6381-6388 (2004); the AAV10 genome is provided in Mol. Ther., 13(1): 67-76 (2006); the AAV11 genome is provided in Virology, 330(2): 375-383 (2004); the AAV12 genome is provided in J. Virol. 2008 February; 82(3): 1399-406; and the AAV13 genome is provided in J. Virol. 2008; 82: 8911. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka (Current Topics in Microbiology and Immunology, 158: 97-129 (1992)).

AAV possesses unique features that make it attractive as a vector for delivering foreign DNA to cells, for example, in gene therapy. AAV infection of cells in culture is noncytopathic, and natural infection of humans and other animals is silent and asymptomatic. AAV infects many mammalian cells allowing the possibility of targeting many different tissues in vivo. AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. In some aspects, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. AAV may be lyophilized and AAV-infected cells are not resistant to superinfection.

In some embodiments, DNA plasmids of the disclosure are provided which comprise rAAV genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV1, AAV2, AAV3, AAV4, AAV5, AAV6, AAV7, AAV8, AAV9, AAV.rh74, AAV.rh8, AAV.rh10, AAV11, AAV12, AAV13, AAV-anc80, AAV-B1, AAV.PHP.EB, or AAVv66. In some aspects, the ITRs in the AAV are from a different AAV serotype. In some aspects, the AAV comprises an ITR or capsid protein which is from a different serotype, i.e., a different serotype than the rest of the vector. For example, in some aspects, AAV2 or AAV2-based ITRs are used in various AAV vectors, not only serotypes which are AAV2 or AAV2-based. In some aspects, various ITRs are interchangeable among the different serotypes of AAV. For example, in some aspects, AAV2 ITRs are interchangeable among the different serotypes of AAV. Thus, in some aspects, AAV2 ITRs are used in a different serotype of AAV vector including, but not limited to, for example, AAV9. In some aspects, AAV2 Rep helper genes are used.

In some aspects, AAV DNA in the rAAV genomes is from any AAV serotype for which a recombinant virus can be derived including, but not limited to, AAV serotypes AAV1, AAV2, AAV3, AAV4, AAV5, AAV6, AAV7, AAV8, AAV9, AAV.rh74, AAV.rh8, AAV.rh10, AAV11, AAV12, AAV13, AAV-anc80, AAV-B1, AAV.PHP.EB, or AAVv66. Other types of rAAV variants, for example rAAV with capsid mutations, are also included in the disclosure. See, for example, Marsic et al., Molecular Therapy 22(11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

Recombinant AAV genomes of the disclosure comprise one or more AAV ITRs flanking at least one KCNQ3-targeted polynucleotide or nucleotide sequence. In some embodiments, the polynucleotide is an miRNA or a polynucleotide encoding the miRNA. In some aspects, the miRNA is administered with other polynucleotide constructs targeting KCNQ3. AAV DNA in the rAAV genomes may be from any AAV serotype for which a recombinant virus can be derived including, but not limited to, AAV serotypes AAV1, AAV2, AAV3, AAV4, AAV5, AAV6, AAV7, AAV8, AAV9, AAV.rh74, AAV.rh8, AAV.rh10, AAV11, AAV12, AAV13, AAV-anc80, AAV-B1, AAV.PHP.EB, or AAVv66. As set out herein above, the nucleotide sequences of the genomes of various AAV serotypes are known in the art.

DNA plasmids of the disclosure comprise rAAV genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV1, AAV2, AAV3, AAV4, AAV5, AAV6, AAV7, AAV8, AAV9, AAV.rh74, AAV.rh8, AAV.rh10, AAV11, AAV12, AAV13, AAV-anc80, AAV-B1, AAV.PHP.EB, or AAVv66. In some aspects, AAV DNA in the rAAV genomes is from any AAV serotype for which a recombinant virus can be derived including, but not limited to, AAV serotypes AAV1, AAV2, AAV3, AAV4, AAV5, AAV6, AAV7, AAV8, AAV9, AAV.rh74, AAV.rh8, AAV.rh10, AAV11, AAV12, AAV13, AAV-anc80, AAV-B1, AAV.PHP.EB, or AAVv66. Other types of rAAV variants, for example rAAV with capsid mutations, are also included in the disclosure. See, for example, Marsic et al., Molecular Therapy 22(11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

In some embodiments, packaging cells are provided. Packaging cells are created in order to have a cell line that stably expresses all the necessary components for AAV particle production. Retroviral vectors are created by removal of the retroviral gag, pol, and env genes. These are replaced by the therapeutic gene. In order to produce vector particles, a packaging cell is essential. Packaging cell lines provide all the viral proteins required for capsid production and the virion maturation of the vector. Thus, packaging cell lines are made so that they contain the gag, pol and env genes. Following insertion of the desired gene into in the retroviral DNA vector, and maintenance of the proper packaging cell line, it is now a simple matter to prepare retroviral vectors

For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing [Samulski et al., 1982, Proc. Natl. Acad. S6. USA, 79:2077-2081], addition of synthetic linkers containing restriction endonuclease cleavage sites [Laughlin et al., 1983, Gene, 23:65-73] or by direct, blunt-end ligation [Senapathy & Carter, 1984, J. Biol. Chem., 259:4661-4666]. The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

In some embodiments, therefore, a method of generating a packaging cell to create a cell line that stably expresses all the necessary components for AAV particle production is provided. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing [Samulski et al., 1982, Proc. Natl. Acad. S6. USA, 79:2077-2081], addition of synthetic linkers containing restriction endonuclease cleavage sites (Laughlin et al., 1983, Gene, 23:65-73) or by direct, blunt-end ligation (Senapathy et al., 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbiol. and Immunol. 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine, 13:1244-1250 (1995); Paul et al., Human Gene Therapy, 4:609-615 (1993); Clark et al., Gene Therapy, 3:1124-1132 (1996); U.S. Pat. Nos. 5,786,211; 5,871,982; 6,258,595; and McCarty, Mol. Ther., 16(10): 1648-1656 (2008). The foregoing documents are hereby incorporated by reference in their entirety herein, with particular emphasis on those sections of the documents relating to rAAV production. The production and use of various types of rAAV are specifically contemplated and exemplified.

Recombinant AAV (i.e., infectious encapsidated rAAV particles) are thus provided herein. In some aspects, genomes of the rAAV lack AAV rep and cap genes; that is, there is no AAV rep or cap DNA between the ITRs of the genomes of the rAAV. In some embodiments, the AAV is a recombinant linear AAV (rAAV), a single-stranded AAV (ssAAV), or a recombinant self-complementary AAV (scAAV).

The disclosure thus provides in some embodiments packaging cells that produce infectious rAAV. In one embodiment, packaging cells are stably transformed cancer cells, such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV, in some aspects, are purified by methods standard in the art, such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In some embodiments, the disclosure includes a composition comprising any of the nucleic acids or any of the vectors described herein in combination with a diluent, excipient, or buffer. In some embodiments, the disclosure provides a composition comprising a vector, e.g., such as a viral vector, as described herein. Thus, compositions comprising delivery vehicles (such as rAAV) described herein are provided. In various aspects, such compositions also comprise a pharmaceutically acceptable carrier. In general, as used herein, “pharmaceutically acceptable carrier” means all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

In various aspects, any composition of the disclosure also comprises other ingredients, such as a diluent, excipients, and/or adjuvant. Acceptable carriers, diluents, excipients, and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

In some aspects, the nucleic acids are introduced into a vector for delivery. In some aspects, the vector for delivery is an AAV or an rAAV. Thus, embodiments of the disclosure include an rAAV genome comprising a nucleotide sequence comprising at least 90% identity to the sequence set forth in any one of SEQ ID NOs: 3-9; the nucleotide sequence set forth in any one of SEQ ID NOs: 3-9; a nucleotide sequence comprising at least 90% identity to the sequence set forth in any one of SEQ ID NOs: 10-16; the nucleotide sequence set forth in any one of SEQ ID NOs: 10-16; a nucleotide sequence that encodes the RNA sequence set forth in any one of SEQ ID NOs: 17-23; or a nucleotide sequence that specifically hybridizes to the KCNQ3 sequence set forth in any one of SEQ ID NOs: 24-30.

In some other aspects, the nucleic acids are introduced into the cell via non-vectorized delivery. Thus, in an embodiment, the disclosure includes non-vectorized delivery of a nucleic acid encoding the KCNQ3-targeting miRNAs. In some aspects, in this context, synthetic carriers able to form complexes with nucleic acids, and protect them from extra- and intracellular nucleases, are an alternative to viral vectors. In some aspects, such non-vectorized delivery includes the use of nanoparticles, extracellular vesicles, or exosomes comprising the nucleic acids of the disclosure. The disclosure also includes compositions comprising any of the constructs described herein alone or in combination.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10³ to about 1×10¹⁴ or more DNase resistant particles (DRP) per ml. Dosages may also be expressed in units of viral genomes (vg) (e.g., 1×10⁷ vg, 1×10⁸ vg, 1×10⁹ vg, 1×⁰¹ vg, 1×10¹¹ vg, 1×10² vg, 1×10³ vg, and 1×10¹⁴ vg, respectively).

In some aspects, therefore, the disclosure provides a method of delivering to a cell or to a subject any one or more nucleic acids comprising a nucleotide sequence comprising at least 90% identity to the sequence set forth in any one of SEQ ID NOs: 3-9; the nucleotide sequence set forth in any one of SEQ ID NOs: 3-9; a nucleotide sequence comprising at least 90% identity to the sequence set forth in any one of SEQ ID NOs: 10-16; the nucleotide sequence set forth in any one of SEQ ID NOs: 10-16; a nucleotide sequence that encodes the RNA sequence set forth in any one of SEQ ID NOs: 17-23; or a nucleotide sequence that specifically hybridizes to the KCNQ3 sequence set forth in any one of SEQ ID NOs: 24-30.

In some aspects, the method comprises administering to a cell or to a subject an AAV comprising any one or more nucleic acids comprising a nucleotide sequence comprising at least 90% identity to the sequence set forth in any one of SEQ ID NOs: 3-9; the nucleotide sequence set forth in any one of SEQ ID NOs: 3-9; a nucleotide sequence comprising at least 90% identity to the sequence set forth in any one of SEQ ID NOs: 10-16; the nucleotide sequence set forth in any one of SEQ ID NOs: 10-16; a nucleotide sequence that encodes the RNA sequence set forth in any one of SEQ ID NOs: 17-23; or a nucleotide sequence that specifically hybridizes to the KCNQ3 sequence set forth in any one of SEQ ID NOs: 24-30.

In yet another aspect, the disclosure provides a method of decreasing expression of the KCNQ3 gene or decreasing the expression of functional KCNQ3 in a cell or a subject, wherein the method comprises contacting the cell or the subject with any one or more nucleic acids comprising a nucleotide sequence comprising at least 90% identity to the sequence set forth in any one of SEQ ID NOs: 3-9; the nucleotide sequence set forth in any one of SEQ ID NOs: 3-9; a nucleotide sequence comprising at least 90% identity to the sequence set forth in any one of SEQ ID NOs: 10-16; the nucleotide sequence set forth in any one of SEQ ID NOs: 10-16; a nucleotide sequence that encodes the RNA sequence set forth in any one of SEQ ID NOs: 17-23; or a nucleotide sequence that specifically hybridizes to the KCNQ3 sequence set forth in any one of SEQ ID NOs: 24-30.

In some aspects, the method comprises delivering the nucleic acids in one or more AAV vectors. In some aspects, the method comprises delivering the nucleic acids to the cell in non-vectorized delivery.

In some aspects, expression of KCNQ3 or the expression of functional KCNQ3 is decreased in a cell or in a subject by the methods provided herein by at least or about 5, about 10, about 15, about 20, about 25, about 30, about 35, about 40, about 45, about 50, about 55, about 60, about 65, about 70, about 75, about 80, about 85, about 90, about 95, about 96, about 97, about 98, about 99, or 100 percent.

In some aspects, the disclosure provides AAV transducing cells for the delivery of nucleic acids encoding the KCNQ3 miRNA as described herein. Methods of transducing a target cell with rAAV, in vivo or in vitro, are included in the disclosure. The methods comprise the step of administering an effective dose, or effective multiple doses, of a composition comprising a rAAV of the disclosure to a subject, including an animal (such as a human being) in need thereof. If the dose is administered prior to development of a seizure υι epileptic disease, or other symptom of the disease or disorder associated with mutant or pathogenic expression of KCNQ3, the administration is prophylactic. If the dose is administered after the development of a seizure or epileptic disease, or other symptom of the disease or disorder associated with mutant or pathogenic expression of KCNQ3, the administration is therapeutic or ameliorative. In embodiments of the disclosure, an effective dose is a dose that alleviates (eliminates or reduces) at least one symptom of the disease or disorder associated with mutant or pathogenic expression of KCNQ3 being treated, that slows or prevents progression of a symptom of the disease or disorder associated with mutant or pathogenic expression of KCNQ3, and/or that results in remission (partial or total) of the symptom(s) of the disease or disorder associated with mutant or pathogenic expression of KCNQ3. In some aspects, the disease or disorder associated with mutant or pathogenic expression of KCNQ3 is developmental and epileptic encephalopathy (DEE).

In some aspects, the disclosure provided non-vectorized delivery of nucleic acids encoding the KCNQ3 miRNA as described herein. In some aspects, the nucleic acids or compositions comprising the nucleic acids are delivered in nanoparticles, extracellular vesicles, or exosomes.

Combination therapies are also contemplated by the disclosure. Thus, the disclosure includes possible combination therapy or therapies comprising one or more other compounds or compositions comprising other RNA inhibitory compounds or small molecule compounds for downregulating KCNQ3 in the treatment of DEE or other condition associated with the mutant or pathogenic expression of KCNQ3. Combination as used herein includes simultaneous treatment or sequential treatments. Combinations of methods of the disclosure with standard medical treatments and supportive care are specifically contemplated, as are combinations of therapies, such as physical and occupational therapies, speech & language therapy, therapy by developmental specialists for their neurodevelopmental delay and autistic symptoms, medications to address behavioral problems (including, but not limited to, alpha-2 adrenergic agonists, antipsychotics, selective serotonin reuptake inhibitors (SSRIs), and the like), medications to address sleep problems (including, but not limited to, melatonin, trazodone, benzodiazepines, doxepine, eszopiclone, lemborexant, ramelteon, suvorexant, zaleplon, zolpidem, and the like) and medications to address seizures and/or EEG abnormalities (including, but not limited to, any of the many anti-seizure medications known in the art including, but not limited to, carbamazepine, eslicarbazepine, ethosuximide, everolimus, gabapentin, lacosamide, oxcarbazepine, lamotrigine, phenobarbital, phenytoin, pregabalin, tiagabine, vigabatrin, valproic acid, acetazolamide, brivaracetam, cannabidiol, cenobamate, clobazam, clonazepam, clorazepate, diazepam, divalproex, felbamate, fenfluramine, lamotrigine, levetiracetam, lorazepam, methsuximide, perampanel, primidone, rufinamide, stiripentol, topiramate valproic acid, or zonisamide) or with other inhibitory RNA constructs are specifically contemplated.

Other combination therapies included in the disclosure are the KCNQ3 miRNAs, as described herein, in combination with other miRNAs, or in combination with U7-snRNA-based gene therapy, a small molecule inhibitor of KCNQ3 expression, oligonucleotides to inhibit KCNQ3 through RNAi or RNAse H or exon skipping mechanisms, U7-snRNA plus a theoretical CRISPR-based gene therapy approach.

Administration of an effective dose of the compositions, including AAV, nanoparticles, extracellular vesicles, and exosomes comprising the compositions and nucleic acids of the disclosure, may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. Route(s) of administration and serotype(s) of AAV components of rAAV (in particular, the AAV ITRs and capsid protein) of the disclosure may be chosen and/or matched by those skilled in the art taking into account the disease state being treated and the target cells/tissue(s), such as cells that express a mutant or pathogenic variant of the KCNQ3 gene resulting in the mutant or pathogenic expression of KCNQ3. In some embodiments, the composition or medicament is formulated for intracerebroventricular injection, intrathecal injection, intramuscular injection, oral administration, subcutaneous, intradermal, or transdermal transport, injection into the blood stream, or for aerosol administration. In some embodiments, the route of administration is intracerebroventricular. In some embodiments, the route of administration is intravenous.

In some aspects, actual administration of rAAV of the present disclosure may be accomplished by using any physical method that will transport the rAAV recombinant vector into the target tissue of an animal. Administration according to the disclosure includes, but is not limited to, injection directly into the brain, the bloodstream, the central nervous system, and/or other organ. Simply resuspending a rAAV in phosphate buffered saline has been demonstrated to be sufficient to provide a vehicle useful for expression in the brain, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as the brain. See, for example, WO 02/053703, the disclosure of which is incorporated by reference herein. Pharmaceutical compositions can be prepared for oral administration, as injectable formulations, or as topical formulations to be delivered to the muscles by subcutaneous, intradermal, and/or transdermal transport. Numerous formulations for both intramuscular injection and transdermal transport have been previously developed and can be used in the practice of the disclosure. The rAAV can be used with any pharmaceutically acceptable carrier for ease of administration and handling.

For purposes of injection, in some aspects, solutions such as sterile aqueous solutions are used. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. In some aspects, proper fluidity is maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

In some aspects, the formulation comprises a stabilizer. The term “stabilizer” refers to a substance or excipient which protects the formulation from adverse conditions, such as those which occur during heating or freezing, and/or prolongs the stability or shelf-life of the formulation in a stable state. Examples of stabilizers include, but are not limited to, sugars, such as sucrose, lactose and mannose; sugar alcohols, such as mannitol; amino acids, such as glycine or glutamic acid; and proteins, such as human serum albumin or gelatin.

In some aspects, the formulation comprises an antimicrobial preservative. The term “antimicrobial preservative” refers to any substance which is added to the composition that inhibits the growth of microorganisms that may be introduced upon repeated puncture of the vial or container being used. Examples of antimicrobial preservatives include, but are not limited to, substances such as thimerosal, 2-phenoxyethanol, benzethonium chloride, and phenol.

The term “transduction” is used to refer to the administration/delivery of one or more of the KCNQ3 targeting constructs, e.g., KCNQ3 miRNA or nucleic acid encoding KCNQ3 miRNA, described herein to a recipient cell either in vivo or in vitro, via a replication-deficient rAAV of the disclosure resulting in decreased expression of KCNQ3 by the recipient cell.

In one aspect, transduction with rAAV is carried out in vitro. In one embodiment, desired target cells are removed from the subject, transduced with rAAV and reintroduced into the subject. Alternatively, syngeneic or xenogeneic cells can be used where those cells will not generate an inappropriate immune response in the subject.

Suitable methods for the transduction and reintroduction of transduced cells into a subject are known in the art. In one embodiment, cells are transduced in vitro by combining rAAV with cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intracerebroventricular, intramuscular, intravenous, subcutaneous and intraperitoneal injection, or by injection into the brain or smooth and cardiac muscle, using e.g., a catheter.

The disclosure provides methods of administering an effective dose (or doses, administered essentially simultaneously or doses given at intervals) of rAAV that comprise DNA that encodes microRNA designed to reduce or inhibit the expression of KCNQ3 to a cell or to a subject in need thereof. In some aspects, the effective dose is therefore a therapeutically effective dose.

In some embodiments, the dose or effective dose of rAAV administered is about 1.0×10¹⁰ vg/kg to about 1.0×10¹⁶ vg/kg. In some aspects, 1.0×10¹⁰ vg/kg is also designated 1.0 E10 vg/kg, which is simply an alternative way of indicating the scientific notation. Likewise, 10¹¹ is equivalent to E11, and the like. In some aspects, the dose of rAAV administered is about 1.0×10¹¹ vg/kg to about 1.0×10¹⁵ vg/kg. In some aspects the dose of rAAV is about 1.0×10¹⁰ vg/kg, about 2.0×10¹⁰ vg/kg, about 3.0×10¹⁰ vg/kg, about 4.0×10¹⁰ vg/kg, about 5.0×10¹⁰ vg/kg, about 6.0×10¹⁰ vg/kg, about 7.0×10¹⁰ vg/kg, about 8.0×10¹⁰ vg/kg, about 9.0×10¹⁰ about 1.0×10¹¹ vg/kg, about 2.0×10¹¹ vg/kg, about 3.0×10¹¹ vg/kg, about 4.0×10¹¹ vg/kg, about 5.0×10¹¹ vg/kg, about 6.0×10¹¹ vg/kg, about 7.0×10¹¹ vg/kg, about 8.0×10¹¹ vg/kg, about 9.0×10¹¹ vg/kg, about 1.0×10¹² vg/kg, about 2.0×10¹² vg/kg, about 3.0×10¹² vg/kg, about 4.0×10¹² vg/kg, about 5.0×10¹² vg/kg, about 6.0×10¹² vg/kg, about 7.0×10¹² vg/kg, about 8.0×10¹² vg/kg, about 9.0×10¹² vg/kg, about 1.0×10¹³ vg/kg, about 2.0×10¹³ vg/kg, about 3.0×10¹³ vg/kg, about 4.0×10¹³ vg/kg, about 5.0×10¹³ vg/kg, about 6.0×10¹³ vg/kg, about 7.0×10¹³ vg/kg, about 8.0×10¹³ vg/kg, about 9.0×10¹³ vg/kg, about 1.0×10¹⁴ vg/kg, about 2.0×10¹⁴ vg/kg, about 3.0×10¹⁴ vg/kg, about 4.0×10¹⁴ vg/kg, about 5.0×10¹⁴ vg/kg, about 6.0×10¹⁴ vg/kg, about 7.0×10¹⁴ vg/kg, about 8.0×10¹⁴ vg/kg, about 9.0×10¹⁴ vg/kg, about 1.0×10¹⁵ vg/kg, about 2.0×10¹⁵ vg/kg, about 3.0×10¹⁵ vg/kg, about 4.0×10¹⁵ vg/kg, about 5.0×10¹⁵ vg/kg, about 6.0×10¹⁵ vg/kg, about 7.0×10¹⁵ vg/kg, about 8.0×10¹⁵ vg/kg, about 9.0×10¹⁵ vg/kg, or about 1.0×10¹⁶ vg/kg.

In some aspects, the dose is about 1.0×10¹¹ vg/kg to about 1.0×10¹⁵ vg/kg. In some aspects, the dose is about 1.0×10¹³ vg/kg to about 5.0×10¹³ vg/kg. In some aspects, the dose is about 2.0×10¹³ vg/kg to about 4.0×10¹³ vg/kg. In some aspects, the dose is about 3.0×10¹³ vg/kg.

In some aspects, an initial dose is followed by a second greater dose. In some aspects, an initial dose is followed by a second same dose. In some aspects, an initial dose is followed by one or more lesser doses. In some aspects, an initial dose is followed by multiple doses which are the same or greater doses.

Methods of transducing a target cell with a delivery vehicle (such as rAAV), in vivo or in vitro, are contemplated. Transduction of cells with an rAAV of the disclosure results in sustained expression of KCNQ3 miRNA sequence. The disclosure thus provides rAAV and methods of administering/delivering rAAV which express KCNQ3 miRNA sequence in the cell(s) in vitro or in vivo in a subject. In some aspects, the subject is a mammal. In some aspects, the mammal is a human. These methods include transducing cells and tissues (including, but not limited to, tissues such as the brain) with one or more rAAV described herein. Transduction may be carried out with gene cassettes comprising cell-specific control elements. The term “transduction” is used to refer to, as an example, the administration/delivery of a nucleic acid comprising a nucleotide sequence encoding a KCNQ3 miRNA sequence, e.g., KCNQ3 miRNA, to a target cell either in vivo or in vitro, via a replication-deficient rAAV described herein resulting in the reduced expression or inhibition of expression of KCNQ3 by the target cell.

The in vivo methods comprise the step of administering an effective dose, or effective multiple doses, of a composition comprising a delivery vehicle (such as rAAV) to a subject (including a human subject) in need thereof. Thus, methods are provided of administering an effective dose (or doses, administered essentially simultaneously or doses given at intervals) of rAAV described herein to a subject in need thereof. If the dose or doses is administered prior to development of a disorder/disease, the administration is prophylactic. If the dose or doses is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival.

In some embodiments, compositions and methods of the disclosure are used in treating, ameliorating, or preventing a disease or disorder associated with expression of a mutant or pathogenic KCNQ3 gene resulting in the mutant or pathogenic expression of KCNQ3 protein. In some aspects, symptoms of such a disease or disorder resulting from the mutant or pathogenic expression of KCNQ3 protein include, but are not limited to, seizures, an epileptic disease or disorder, an intellectual or developmental disability, autism, or an autism spectrum disorder. In some aspects, any of such symptoms are indicative of developmental and epileptic encephalopathy (DEE).

Molecular, biochemical, histological, and functional outcome measures demonstrate the therapeutic efficacy of the products and methods disclosed herein for decreasing the mutant or pathogenic expression of the KCNQ3 mRNA and protein and treating the seizures, epileptic disease or disorder, intellectual or developmental disability, autism, autism spectrum disorder, or DEE resulting from the mutant or pathogenic expression of the KCNQ3 mRNA and protein. Outcome measures include, but are not limited to, reduction or elimination of KCNQ3 mRNA or protein, or its mutant or pathogenic variant(s), in affected tissues. The lack of expression of KCNQ3 and/or the downregulation of expression of the mutant or pathogenic KCNQ3 mRNA or protein in the cell is detected by measuring the level of KCNQ3 protein by methods known in the art including, but not limited to, RT-PCR, QRT-PCR, RNAscope, Western blot, immunofluorescence, or immunohistochemistry in brain biopsied before and after administration of the microRNA or the rAAV comprising the microRNA to determine the improvement.

In some embodiments, the level of KCNQ3 gene expression or protein expression in a cell of the subject is decreased after administration of the nucleic acid encoding the KCNQ3 miRNA or the vector, e.g., rAAV, comprising the nucleic acid encoding the KCNQ3 miRNA as compared to the level of KCNQ3 gene expression or protein expression before administration of the nucleic acid encoding the KCNQ3 miRNA or the vector, e.g. rAAV. In some aspects, expression of a KCNQ3 is decreased by at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%. In various aspects, the number, frequency, or intensity of seizures or incidences of epilepsy show an improvement by at least about 2%, at least about 5%, at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%.

Another outcome measure includes assessing the level of membrane-bound KCNQ3 protein before and after treatment, since it is shown to be elevated as a result of mutation in adult mice. Thus, a positive therapeutic outcome for treatment with the products and methods of the disclosure is a reduction in the level of membrane-bound KCNQ3 after administration of KCNQ3 miRNA (or AAV comprising the KCNQ3 miRNA) as compared to before administration of the KCNQ3 miRNA (or AAV comprising the KCNQ3 miRNA).

Another outcome measure includes examination of the intracranial EEG after administration of KCNQ3 miRNA (or AAV comprising the KCNQ3 miRNA) as compared to before administration of the KCNQ3 miRNA (or AAV comprising the KCNQ3 miRNA). There is significant epileptiform activity in mutant mouse pups in their second postnatal week, which has similarities to the electroclinical features of children with R231 pathogenic variants of KCNQ3. Thus, it is expected that products and methods of the disclosure improve or decrease epileptiform activity.

Another outcome measure includes examination of rest activity behavior after administration of KCNQ3 miRNA (or AAV comprising the KCNQ3 miRNA) as compared to before administration of the KCNQ3 miRNA (or AAV comprising the KCNQ3 miRNA). Mice carrying pathogenic R231 variants are moderately hyperactive. Thus, it is expected that products and methods of the disclosure improve or decrease hyperactivity.

Administration of an effective dose of a nucleic acid, viral vector, or composition of the disclosure may be by routes standard in the art including, but not limited to, intracerebroventricular, intrathecal, intravenous, intracranial, oral, buccal, nasal, intraosseous, intramuscular, parenteral, intravascular, pulmonary, intraocular, rectal, or vaginal. In some aspects, an effective dose is delivered by a systemic route of administration, i.e., systemic administration. Systemic administration is a route of administration into the circulatory system so that the entire body is affected. Such systemic administration, in various aspects, takes place via enteral administration (absorption of the drug through the gastrointestinal tract) or parenteral administration (generally via injection, infusion, or implantation). In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration and serotype(s) of AAV components of the rAAV (in particular, the AAV ITRs and capsid protein) of the disclosure, in various aspects, are chosen and/or matched by those skilled in the art taking into account the condition or state of the disease or disorder being treated, the condition, state, or age of the subject, and the target cells/tissue(s) that are to express the nucleic acid or protein.

In particular, actual administration of delivery vehicle (such as rAAV) may be accomplished by using any physical method that will transport the delivery vehicle (such as rAAV) into a target cell of an animal. Administration includes, but is not limited to, injection into the brain, the nervous system, the liver, or the bloodstream. Simply resuspending a rAAV in phosphate buffered saline has been demonstrated to be sufficient to provide a vehicle useful for expression in the brain, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as neurons. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof. [00126]“Treating” includes ameliorating, reducing, or inhibiting one or more symptoms of a seizure or an epileptic episode including, but not limited to, decreasing or eliminating seizures, decreasing seizure intensity, and/or decreasing the number of seizures. Treating also includes improvements in or the elimination of various symptoms associated with the expression of the KCNQ3 variants (i.e., the KCNQ3 pathogenic protein(s)) disclosed herein including, but not limited to, developmental delay, cognitive dysfunction, autism, behavioral problems, epilepsy, hypotonia, and/or strabismus.

The disclosure also provides a kit comprising a nucleic acid, vector, or composition of the disclosure or produced according to a process of the disclosure. In the context of the disclosure, the term “kit” means two or more components, one of which corresponds to a nucleic acid, vector, or composition of the disclosure, and the other which corresponds to a container, recipient, instructions, or otherwise. A kit, therefore, in various aspects, is a set of products that are sufficient to achieve a certain goal, which can be marketed as a single unit.

The kit may comprise one or more recipients (such as vials, ampoules, containers, syringes, bottles, bags) of any appropriate shape, size and material containing the nucleic acid, vector, or composition of the disclosure in an appropriate dosage for administration (see above). The kit may additionally contain directions or instructions for use (e.g. in the form of a leaflet or instruction manual), means for administering the nucleic acid, vector, or composition, such as a syringe, pump, infuser or the like, means for reconstituting the nucleic acid, vector, or composition and/or means for diluting the nucleic acid, vector, or composition.

In some aspects, the kit comprises a label and/or instructions that describes use of the reagents provided in the kit. The kits also optionally comprise catheters, syringes or other delivering devices for the delivery of one or more of the compositions used in the methods described herein.

The disclosure also provides kits for a single dose of administration unit or for multiple doses. In some embodiments, the disclosure provides kits containing single-chambered and multi-chambered pre-filled syringes.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning.

Unless otherwise indicated, the term “at least” preceding a series of elements is to be understood to refer to every element in the series. Those skilled in the art will recognize, or be able to ascertain using no more than routine experimentation, many equivalents to the specific embodiments of the disclosure described herein. Such equivalents are intended to be encompassed by the disclosure.

The term “and/or” wherever used herein includes the meaning of “and”, “or” and “all or any other combination of the elements connected by said term.”

The term “about” or “approximately” as used herein means within 20%, preferably within 10%, and more preferably within 5% of a given value or range. It includes, however, also the concrete number, e.g., about 10 includes 10.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein, “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this disclosure is not limited to the particular methodology, protocols, material, reagents, and substances, etc., described herein and as such can vary. The terminology used herein is for the purpose of describing particular embodiments only, and is not intended to limit the scope of the subject matter of the disclosure, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material.

A better understanding of the disclosure and of its advantages will be obtained from the following examples, offered for illustrative purposes only. The examples are not intended to limit the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional aspects and details of the disclosure will be apparent from the following examples, which are intended to be illustrative rather than limiting.

### Example 1

### Materials and Methods

Study Design. The objective of the study was to explore new strategies to treat epilepsy and related conditions resulting from the mutant or pathogenic expression of KCNQ3. More specifically, the objective of the study was to explore new strategies to treat epilepsy and related conditions resulting from inherited or de novo missense mutations in the KCNQ3 gene. In some aspects, such inherited mutations include the gain-of-function mutations (R230C, R230H, R230S, and R227Q of KCNQ3) which cause DEE. Thus, the products and methods of the disclosure are designed to interfere with the expression of mutant or pathogenic KCNQ3 gene mutations which result in the expression of various mutant or pathogenic form(s) of KCNQ3 protein.

An RNAi approach was taken to decrease the expression of a pathogenic variant (KCNQ3-R230H) responsible for a form of developmental and epileptic encephalopathy (DEE), and the approached was reduced to practice using a mouse model expressing the orthologous genotype (i.e. Kcnq3^{R231H/+}), which is a mouse model of KCNQ3 developmental and epileptic encephalopathy. Mutations in humans that reliably result in DEE in the heterozygous condition include R230C, R230H, R230S, and R227Q, and all of these mutations are included for treatment with the products and methods disclosed herein. R231 is the mouse residue corresponding to R230 in humans and, thus, the mouse model expressing the orthologous genotype (i.e. Kcnq3^{R231H/+}) disclosed herein was developed and tested as disclosed herein.

Because mice that completely lack Kcnq3 from conception are only very mildly impaired with respect to overt clinical phenotypes or seizures (Soh et al. (2014) J Neurosci. 34: 5311-21), the approach taken was to create an RNAi construct (microRNA (miRNA)) that targets both mutant and wildtype copies of Kcnq3 mRNA. The hypothesis was that the reduction of wildtype Kcnq3 mRNA would have little or no detrimental effect on the subject, whereas reduction of the mutant Kcnq3 mRNA would significantly diminish phenotypic features that model the human disease resulting from the mutant.

Design and cloning of artificial microRNAs targeting KCNQ3. All design rules for artificial miRNAs were followed as described (Wallace, et al PMID 29387734; Wallace et al., Mol Ther Methods Clin Dev, 2018. 8: p. 121-130), including: 22-nucleotide mature miRNA length, antisense complementarity to the human and rodent target mRNA (KCNQ3/Kcnq3), less than 60% GC content of the mature duplex, and guide-strand biasing, such that the last 4 nucleotides of the antisense 5′ end were A:U rich, and the last 4 nucleotides of the antisense 3′ end were G:C rich. MiRNAs were cloned into a U6T6 expression vector (Boudreau, R. L., et al., Rapid Cloning and Validation of MicroRNA Shuttle Vectors: A Practical Guide., in RNA Interference Methods, S. Q. Harper, Editor. 2011, Humana Springer Press. p. 19-37). After in vitro testing (using luciferase assay as described below), lead candidate U6.miRNAs were cloned into a self-complementary proviral AAV plasmid (scAAV) containing a CMV-driven eGFP reporter. Self-complementary AAV serotype 9 (scAAV9) viruses were generated and titered by Andelyn Biosciences (Columbus, OH). Vector titer calculations were performed using linear DNA standards.

HEK293 Cell Culture. HEK293 cells were grown using DMEM (Gibco) medium supplemented with 20% FBS (Corning), 1% L-glutamine (Gibco) and 1% Penicillin-Streptomycin (Gibco). Transfected cells were grown in the same DMEM medium but lacking Penicillin-Streptomycin.

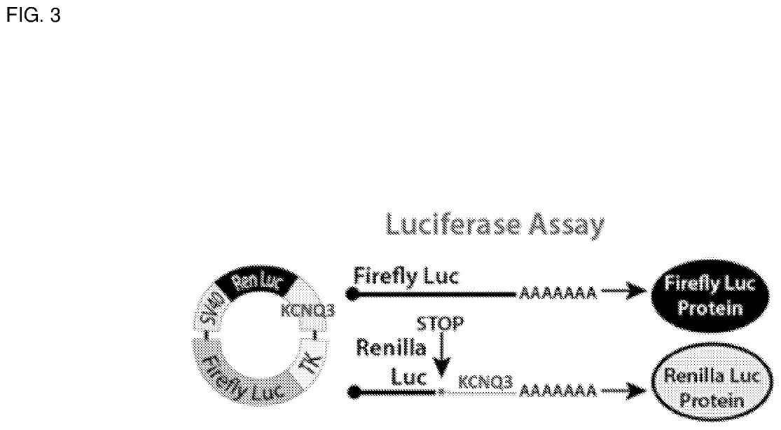

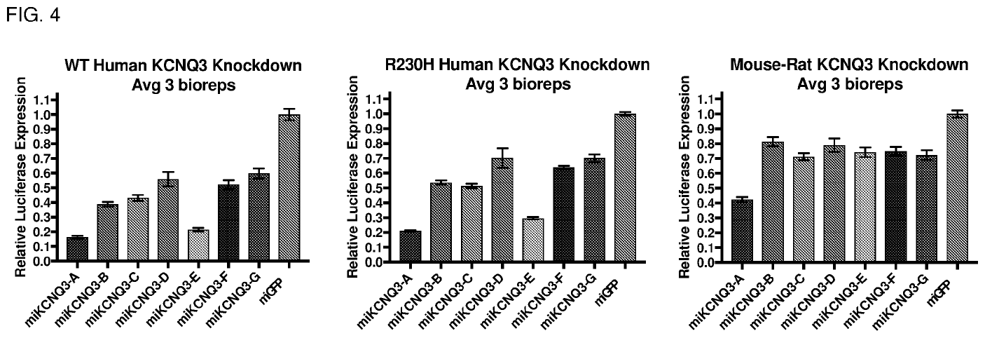

Dual luciferase assay. The dual luciferase plasmids were created in the Psicheck2 vector (Promega), with firefly luciferase serving as a control, and human KCNQ3 or mouse/rat Kcnq3 target regions cloned downstream of the Renilla luciferase stop codon (FIG. 3). HEK293 cells were cotransfected (Lipofectamine 2000, Invitrogen) with the appropriate reporter and an individual U6.miRNA expression plasmid in a 1:5 molar ratio. KCNQ3/Kcnq3 silencing was determined 48 hours after transfection, using the Dual-Luciferase Reporter Assay System (Promega). Individual assays were performed in triplicate, and three independent experiments were averaged. Results of the assay are shown in FIG. 4, and are presented as the mean ratio of Renilla to firefly luciferase±SEM.

Kcnq3 R231H gain-of-function (GoF) mutation mice. C57BL/6J and FVB/NJ mice were purchased from The Jackson Laboratory and maintained by brother-sister matings in the vivarium at Columbia. Kcnq3^{R231H} mice were developed in the transgenic core at the Columbia Herbert Irving Comprehensive Cancer Center by using CRISPR/Cas9 mutagenesis with a donor oligonucleotide in C57BL/6J zygotes with the sgRNA 5′-GCAGGAUCUGCAGGAAGCGA-3′ (SEQ ID NO: 38) to change the Arg 231 CGC codon to CAC His and also to eliminate a PstI restriction enzyme site for convenient genotyping. Founder mice were crossed to wildtype C57BL/6J and thereafter backcrossed to wildtype C57BL/6J to maintain the line. For RNAi studies, Kcnq3R231^{H/+} heterozygous males were mated to wildtype FVB/NJ to make the F1 hybrid population segregating the Kcnq3^{R231H} mutation and used for viral injection, EEG testing, and assessment of mRNA and protein abundance (Sands et al., www.aesnet.org/abstractslisting/kcnq3-gain-of-function-mouse-model—electroclinical-and-behavioral-phenotype). All mouse procedures were approved by Columbia University's Institutional Animal Care and Use Committee and were performed in accordance with the National Institute of Health guide for the care and use of laboratory animals.

scAAV9 treatment of mouse pups. On the day after F1 hybrid pups were born, a maximum of 10 μl of either scAAV9-U6-miKcnq3-A-GFP virus (8.6×10¹⁰ vg/mouse) or scAAV9-eGFP virus (7.3×10¹⁰ vg/mouse) was delivered by intracerebroventricular (icv) injection under hypothermia anesthesia by using a sterile Hamilton syringe. Pups were place back with their mothers in their home cages and held for phenotypic assessments.

EEG studies. After 40 days of age, mice were surgery implanted with subdural electrodes as previously described (PMID: 32577763) and allowed to recover for at least 48 hours before EEG recordings. Recordings were acquired on a Quantum 128 amplifier and Natus Neuroworks software (Natus, Inc), and EDF format files were exported and analyzed using Assyst version 3 software (Kaoskey, Inc). SWD detection and processing was performed using automated algorithms only. The following settings were used for initial detection: Processing window—1.0 s, 0.25 s steps; Frequency band—14 Hz-23 Hz, 20 FC samples; SBI processing set to smoothed SBI, 2 passes; Threshold definition—relative position 0.1; Event processing—events shorter than 0.45 s were removed, larger than 2.0 s were joined. The following parameters were then used for SWD processing: Event time refinement window size—0.5 s, Running beat spectrum calculation parameters—spectrum length—1.0 s, window size—0.05 s; SWD identification parameters—main frequency range 6.5 Hz-10 Hz, 4 beat spectrum peaks for SWD, first peak relative amplitude average threshold—0.35, number of first peaks—3. Following these algorithms, SWD self-sorted into positive and negative lists, and a manual review was done to eliminate false negative and false positive events. No manual event joining or trimming was done for this analysis. Event lists were uploaded for compilation, genotype-treatment decoding and statistical analysis using Microsoft Excel and JMP 16 software.

RNA Extraction. Brain tissue was flash frozen with 2-Methylbutane and stored at −80° C. Samples were homogenized using a dounce and RNA was isolated using TRIzol Reagent (ThermoFisher, Waltham, MA, Cat #15596018). RNA was converted to cDNA using Invitrogen SuperScript III First-Strand Synthesis System (Carlsbad, CA, Cat #18080051).

Quantitative RT-PCR analysis. Quantitative RT-PCR analysis was done on a QuantStudio 5 RealTime PCR system (ThermoFisher Scientific, Inc) with the following primers for Kcnq3 (5′-CACCGTCAGAAGCACTTTGAG-3′ (SEQ ID NO: 39), 5′-CCTTTAGTATTGCTACCACGAGG-3′ (SEQ ID NO: 40)), Actb (5′-GGCTGTATTCCCCTCCATCG-3′ (SEQ ID NO: 41), 5′-CCAGGTAACAATGCCATGT-3′ (SEQ ID NO: 42)), and eGFP(5′-ACGTAAACGGCCACAAGTTC-3′ (SEQ ID NO: 43), 5′-CTGGGTGCTCAGGTAGTGGT-3′ (SEQ ID NO: 44)). For data analysis, threshold Cot (Ct) values were determined for endogenous Kcnq3 and Actb mRNA, and for eGFP mRNA introduced exogenously by the virus. ΔCt was then calculated for Kcnq3 and for eGFP by subtracting Actb from each as the endogenous standard, and a transduction-specific ΔCt for Kcnq3 was calculated by further subtracting eGFP to enrich analysis for transduced cells. Statistical assessment was done by converting ΔCt values to non-parametric and least squares regression using JMP 16 software.

Western blot analysis. Dissected mouse brain tissue was snap frozen in liquid nitrogen and stored at −80° C. until the time of extraction. Tissue was thawed on ice and homogenized using a motorized pestle in RIPA buffer containing both protease and phosphatase inhibitor cocktail (Roche). Samples were centrifuged and the resulting supernatant was collected and quantified using BCA method (Pierce) with BSA as standard. Using Xcell Surelock Mini Cell system, a total of 15 ug of protein lysate per sample was loaded onto a 4-12% SDS-PAGE gels and subsequently transferred to PVDF membrane. The membranes were incubated with primary antibodies—KCNQ3—1:1000 (Synaptic systems—Kv7.3—368003); ACTB—1:15,000 (Santa Cruz Biotechnology: sc-47778) overnight at 4° C., followed by incubation with secondary HRP-conjugated goat anti-rabbit (1:10,000) (Proteintech—SA00001-2) for 1 hour at room temperature. Signals were developed using Amersham ECL Western Blotting Detection Reagent (GE Healthcare, RPN2106) and visualized using western blot imaging system (Azure Biosystems, Azure C400).

### Example 2

Design and Functional In Vitro Screening of Artificial microRNAs Targeting Conserved Regions on Human and Rodent KCNQ3 Transcripts

Artificial microRNAs of the disclosure were designed using an algorithm as described by Wallace et al. ((2017) Mol Ther Methods Clin Dev. December 24, 8:121-130; also see Boudreau et al. (2011) “Rapid Cloning and Validation of MicroRNA Shuttle Vectors: A Practical Guide.” RNA Interference Methods. Ed. S. Q. Harper. Humana Springer Press, 2011, pages 19-37). In brief, all microRNAs contain processing sites for the RNAse Ill enzymes Drosha and Dicer, yielding a mature, 22 nucleotide (nt) duplex RNA containing 2 nt 3′ overhangs on both strands (FIGS. 1-2). The antisense guide strand of the microRNAs become incorporated into the RNA-Induced Silencing Complex (RISC), where they direct cellular gene silencing machinery to cleave target mRNAs, in this case human KCNQ3 or rodent Kcnq3.

To identify microRNAs with the characteristics listed above, human KCNQ3 cDNA was used as a query sequence (SEQ ID NO: 1). The longest full-length KCNQ3 transcript listed on ENSEMBL is 11,583 nucleotides long, containing a 563 nt and 8,401 nt 3′ UTR (ENSEMBL transcript ID ENST00000388996.10; KCNQ3-201). The open reading frame (ORF) is 2,619 nt long (ENSEMBL CCDS34943; SEQ ID NO: 1). Because species conservation (human, mouse, and rat) were including in designing the miRNA constructs, only the ORF was used as query sequence, since protein coding regions typically contain the highest amount of conservation among species. Thus, using the 2,619 nt human KCNQ3 ORF as the query sequence (SEQ ID NO: 1), 152 candidate microRNAs were identified that matched the desired criterion as set out above. The microRNA are not allele specific for only known mutations, but instead target both mutant and wild-type KCNQ3. Allele specificity was not required because only patients having KCNQ3 mutations require treatment to decrease expression of the mutant or pathogenic form of KCNQ3 protein.

The human KCNQ3 ORF was aligned with that of rat and mouse Kcnq3 ORFs to identify microRNA binding sites located in conserved regions of each transcript. Seven miRNA, i.e., miKCNQ3-A-G, were identified and constructed. All candidate microRNAs were cloned into a U6T6 plasmid containing a U6 promoter and RNA polymerase III termination signal (TTTTTT; SEQ ID NO: 45) and sequence verified. See FIG. 2 for binding sites, sequence alignments, and folded primary miRNA transcripts.

### Example 3

### Reduction or Inhibition of KCNQ3 Protein Levels In Vitro

To measure silencing of human or rodent KCNQ3, luciferase reporter plasmids containing human KCNQ3 or rodent Kcnq3 sequences as the 3′ UTR of Renilla luciferase were constructed. The reporter plasmid contained a second gene, Firefly luciferase, which was used as a normalization control (FIG. 3).

To develop an effective miRNA reagent targeting Kcnq3 mRNA, the potential seven KCNQ3/Kcnq3-specific miRNAs were tested for efficacy against target after heterologous expression in HEK293 cells in vitro using a luciferase assay. More specifically, to perform the luciferase assay screen, HEK293 cells were transfected with U6.miKCNQ3 plasmids (miKCNQ3A-G), non-targeting control plasmid (miGFP), and the KCNQ3 luciferase reporter plasmid. Luciferase activity was measured 48 hours later (FIG. 4).

All seven miRNAs tested resulted in significant decrease of the human target mRNA. However, only sequence A (miKCNQ3-A) triggered silencing of the rodent Kcnq3 sequences attached to Renilla luciferase. This was unexpected but not unprecedented. The 5′ end of the rodent Kcnq3 cDNA is highly GC rich with repetitive sequences. It is possible that attaching these difficult sequences (i.e., sequences that have a high GC content are hard to clone and often result in deletions) as the 3′ UTR of Renilla luciferase could impact mRNA folding, thereby making target sites inaccessible to the microRNAs and endogenous silencing machinery. Because sequence A triggered silencing of both human and rodent KCNQ3/Kcnq3 transcripts in this initial testing, it was selected as an initial lead and cloned as a U6-miKQNC3-A into the scAAV9 proviral plasmid (scAAV9-miKCNQ3), which also contained a separate CMV-eGFP reporter gene.

Self-complementary AAV9 vectors were generated, purified, and titered by Andelyn Biosciences for transfection into HEK cells for large-scale virus production of AAV9 vector particles, purification and concentration. scAAV9-eGFP virus was similarly produced and used as control virus for in vivo studies.

### Example 4

### Characterization of a Mouse Model of Epilepsy (Kcnq3^{R231H/+} Mice)

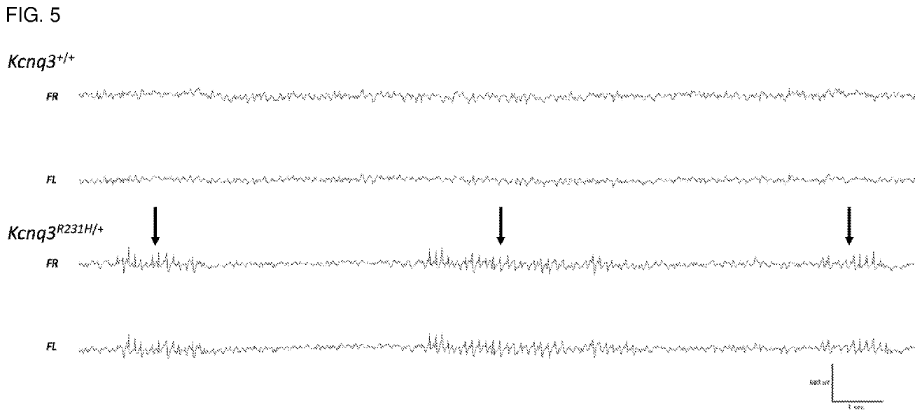

In the characterization of clinically-relevant phenotypic features of the Kcnq3^{R231H/+} mice, it was determined that heterozygotes, but not wildtype littermates, have a type of generalized epilepsy in the form of frequent, spontaneous spike-wave discharges (SWD) in the electroencephalogram (EEG) (FIG. 5). EEG is considered to be the “gold standard” for seizures, and beyond epilepsy it is also one of the strongest, clearest and quantitative measures among all neurobehavioral preclinical behaviors.

Prior to in vivo testing of scAAV9-miKcnq3-A, basic molecular features of the Kcnq3^{R231H/+} mice mutant mice were characterized. Heterozygote Kcnq3^{R231H/+} mice demonstrate a significantly decreased threshold (increased susceptibility) to electrically-induced maximal seizures, compared to wildtype littermates, indicating a general propensity towards seizures. Each of these electroclinical features is quantitative and reproducible, representing strong and clinically-relevant endpoints against which to measure efficacy of new therapies.

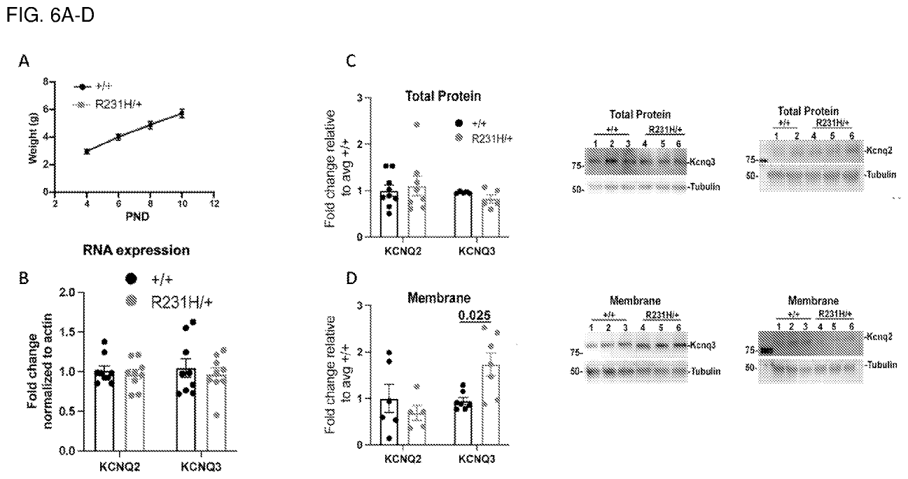

First, it was determined that total Kcnq3 mRNA and protein levels in the heterozygous mouse brain are not significantly different from that of wildtype littermates (FIG. 6A-D). Similarly, no differences were seen with Kcnq2 mRNA and protein levels. Kcnq2 is the primary subunit in the heterotetrameric Kcnq3 ion channel. These results are consistent with the fact that Kcnq3^{R231H} encodes a gain-of-function mechanism, as previously determined in heterologous expression studies (Miceli et al., Front Physiol. 2020 Sep. 4; 11:1040).

An increase of membrane-bound Kcnq3 (but not Kcnq2) was observed. Without being bound by theory, this increase could possibly be the result of compensation due to altered physiology, which may be due to some increased trafficking as this has been reported for the KCNQ1 channel (Huang et al., J Biol Chem, 2021. 296: p. 100423).

### Example 5

### Reduction or Inhibition of KCNQ3 Protein Levels In Vivo

Experiments were designed to test in vivo the scAAV9-miKcnq3 vector for efficacy against the spontaneous seizure phenotype. Matings were setup between FVB/NJ females (The Jackson Laboratory) and C57BL/6J-Kcnq3R231^{H/+} males (Columbia Herbert Irving Comprehensive Cancer Center) to produce roughly equal numbers of F1 hybrid Kcnq3R231^{H/+} (heterozygous) and Kcnq3^{+/+} (wildtype) littermates, genetically identical except for the Kcnq3 genotype. The F1 hybrid rather than the inbred C57BL/6J strain background was used because of the known hybrid vigor and litter sizes, greatly facilitating study logistics, while maintaining a genetically uniform background in the test population (F1 hybrids are identical genotypically to each other, having one chromosomal copy from each parent strain). Mouse pups were genotyped on postnatal day 0 and each mouse was treated on postnatal day 1 by unilateral intracerebral injection with 10 μl of control virus (scAAV9-CMV-eGFP; 7.3×10¹⁰ g), experimental virus (8.6×10¹⁰ vg), or normal saline. Eight heterozygotes were treated with control virus, and 10 heterozygotes were treated with experimental virus. In addition, 4 wildtype mice were treated with the same amount of experimental virus, and 3 wildtype mice were treated with saline.

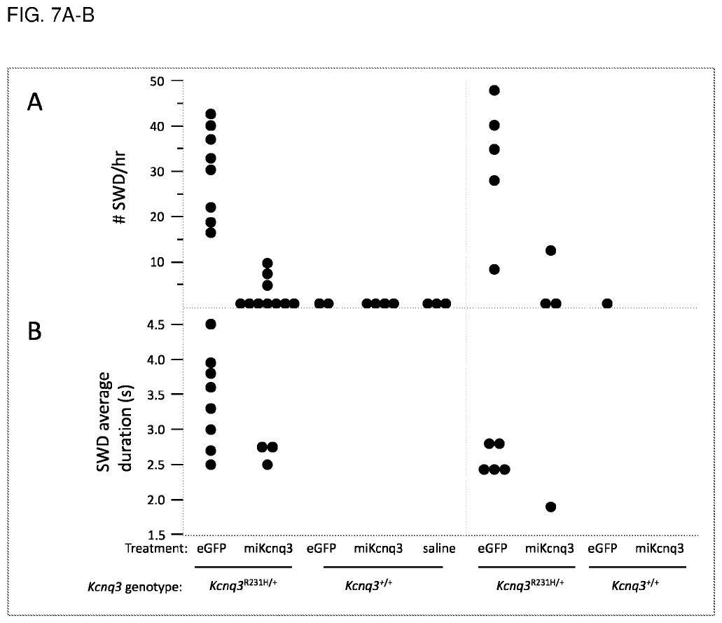

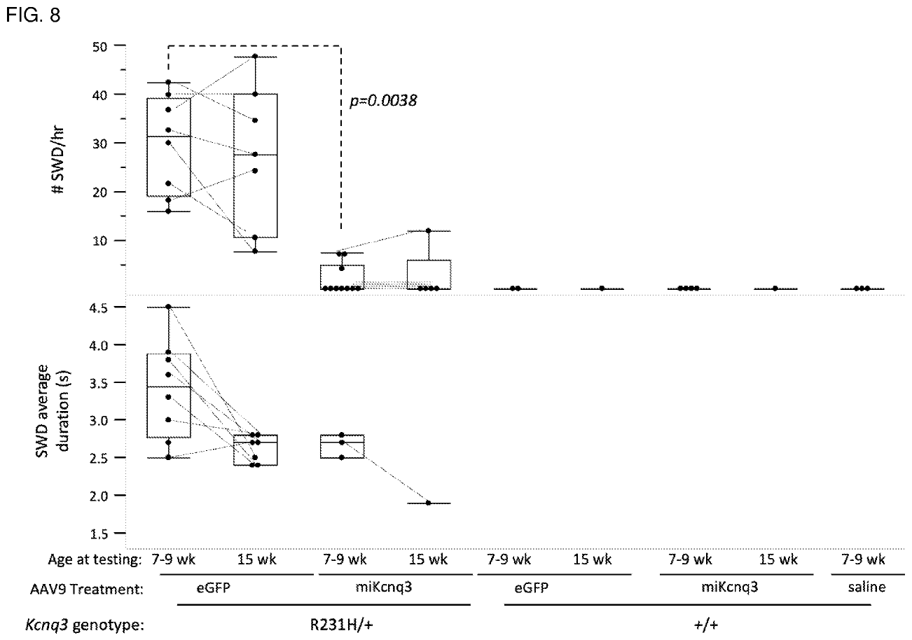

Adult mice were surgically implanted with recording electrodes after postnatal day 40, and recorded by video-EEG between 47 and 61 days postnatal for a continuous 24-hour period (see Table 3 and FIG. 8). Three heterozygote mice treated with experimental virus; five heterozygote mice treated with control virus; and one wildtype mouse treated with control virus were held for an additional time period and video-EEGs were recorded at about 15 weeks postnatal. The results of these experiments are summarized in Table 3 and FIGS. 7 and 8.

TABLE 3
Generalized seizure activity in miKcnq3 vs. control treated mice

| | Kcnq3 | | | | |
|---|---|---|---|---|---|
| i.c.v. | genotype | +/+ | R231H/+ | R231H/+ | R231H/+ |
| treatment | Age at | # mice (no | | avg. SWD/ | avg. SWD |
| (PND1) | recording | SWD/SWD) | | hr ± SEM | duration(s) ± SEM |
| AAV9-miKcnq3 | 7-9 wks | 0/4 | 3/7 | 1.8 ± 1.0 | 2.6 ± 0.05 |
| AAV9-eGFP | ″ | 0/4 | 8/8 | 29.7 ± 3.6 | 3.4 ± 0.02 |
| saline | ″ | 0/4 | n.d. | | |
| AAV9-miKcnq3 | 15 wks | 0/1 | 1/5 | 2.4 ± 2.4 | 1.9 |
| AAV9-eGFP | ″ | 0/1 | 7/7 | 27.4 ± 5.6 | 2.6 ± 0.1 |
| saline | ″ | n.d | n.d. | | |

Results showed that only 3 of 7 mutant mice (Kcnq3^{R231H/+}) had any SWD when treated with scAAV9-miKcnq3, compared with 8/8 mice with SWD when treated with the control eGFP virus. Although 3 mutant mice (Kcnq3^{R231H/+}) did have SWD, the incidence was average fold lower than in control virus treated mice. The average incidence of SWD was 15-fold greater in control virus-treated mice compared with miKcnq3-treated mice.

These results indicate that scAAV9-miKcnq3-A was extremely effective at decreasing the incidence, duration, and severity of this form of seizure in mice carrying the orthologue of a pathological clinical variant of KCNQ3.

FIG. 7A-B shows that there was a significant decrease in SWD incidence (FIG. 7A) and SWD average duration (FIG. 7B) in adult Kcnq3^{R231H/+} mice transduced with scAAV9-miKCNQ3 as neonates. FIG. 8 shows a decrease in SWD incidence (top panel) and a decrease in SWD duration (bottom panel) in Kcnq3^{R231H/+} adult mice transduced with scAAV9-miKcnq3-A as neonates. Dotted lines show the same mice tested at both ages. The p-value shown is based on a 1-tailed Fisher Exact Test.

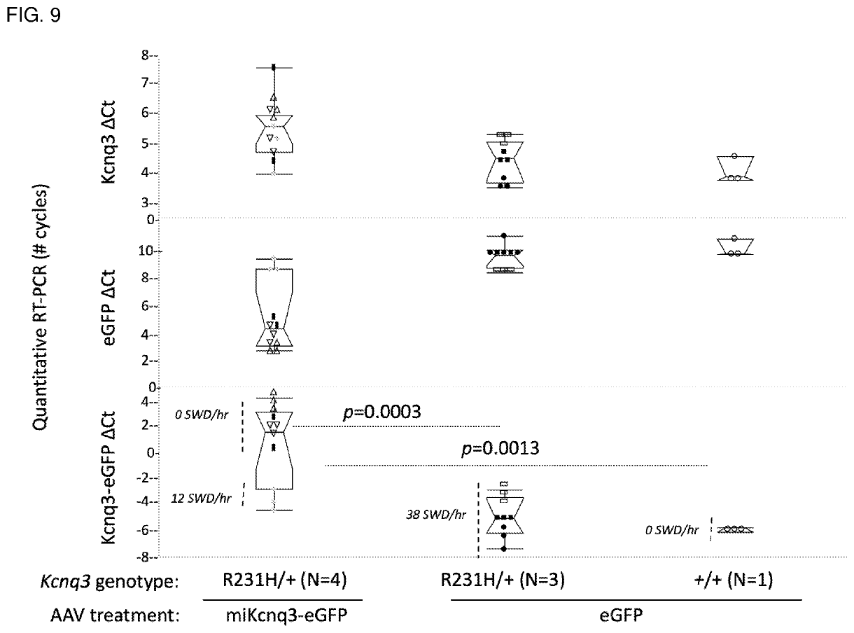

Following assessment of SWD, brain samples were collected from 5 heterozygotes treated with experimental virus, 3 heterozygotes treated with control virus, and 1 wildtype treated with control virus to examine Kcnq3 mRNA levels following miKcnq3 or eGFP treatment. The results clearly show the reduced expression of Kcnq3 mRNA relative to eGFP associated with miKcnq3 treatment (FIG. 9).

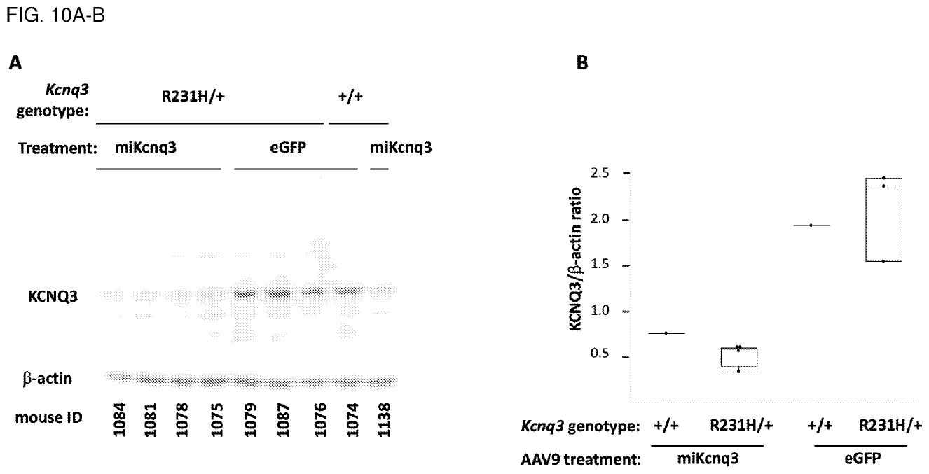

Post-EEG testing, protein was extracted from brain samples and Western blot analysis (FIG. 10A) and densitometry quantification (FIG. 10B) was carried out to measure KCNQ3 protein using an antibody to KCNQ3 and an antibody to β-actin as a loading control (FIG. 10A-B). There was a significant reduction in the amount of KCNQ3 protein in scAAV9-miKcnq3-A treated mice compared with mice treated with scAAV9-eGFP control virus, irrespective of whether the treated mice were heterozygous mutant or wildtype (+/+) littermates, as would be expected for RNAi that targets the mRNA irrespective of the mutant allele.

This study showed that the scAAV9-miKcnq3-A vector was effective in reducing Kcnq3 mRNA and protein expression and was extremely effective at decreasing the incidence, duration, and severity of this form of seizure in mice carrying the orthologue of a pathological clinical variant of KCNQ3.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment.

Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

We claim:

1. A nucleic acid encoding a Potassium Channel, Voltage Gated KQT-Like Subfamily Q, Member 3 (KCNQ3)-targeting microRNA (miRNA) comprising:
(a) a nucleotide sequence comprising at least 90% identity to the sequence set forth in any one of SEQ ID NOs: 3-9;
(b) the nucleotide sequence set forth in any one of SEQ ID NOs: 3-9;
(c) a nucleotide sequence that encodes the RNA sequence set forth in any one of SEQ ID NOs: 17-23; or
(d) a nucleotide sequence that specifically hybridizes to the KCNQ3 sequence set forth in any one of SEQ ID NOs: 24-30.

2. The nucleic acid of claim 1 further comprising a promoter and/or enhancer.

3. The nucleic acid of claim 2, wherein the promoter and/or enhancer is any of a U6 promoter and/or enhancer, a U7 promoter and/or enhancer, a tRNA promoter and/or enhancer, an H1 promoter and/or enhancer, a CMV promoter and/or enhancer, a minimal CMV promoter and/or enhancer, a T7 promoter and/or enhancer, an EF1-alpha promoter and/or enhancer, a minimal EF1-alpha promoter and/or enhancer, an unc45b promoter and/or enhancer, a CK1 promoter and/or enhancer, a CK6 promoter and/or enhancer, a CK7 promoter and/or enhancer, a CK8 promoter and/or enhancer, a ubiquitous promoter and/or enhancer, a neuronal-specific promoter and/or enhancer, or a brain-specific promoter and/or enhancer.

4. The nucleic acid of claim 3 or 4, wherein the promoter and/or enhancer is U6.

5. The nucleic acid of any one of claims 2-4 comprising:
(a) a nucleotide sequence comprising at least 90% identity to the sequence set forth in any one of SEQ ID NOs: 10-16; or
(b) the nucleotide sequence set forth in any one of SEQ ID NOs: 10-16.

6. The nucleic acid of claim 3, wherein the brain-specific promoter and/or enhancer is human Synapsin1 (hSyn1), neuron-specific enolase (Nse), MeCP2, mDLX, mDLX5/6, or calmodulin-dependent kinase II (CaMKII or Camk2a).

7. An adeno-associated virus comprising the nucleic acid of any one of claims 1-6.

8. The adeno-associated virus of claim 7, wherein the virus lacks rep and cap genes.

9. The adeno-associated virus of claim 7 or 8, wherein the virus is a recombinant AAV (rAAV) or a self-complementary recombinant AAV (scAAV).

10. The adeno-associated virus of any one of claims 7-9, wherein the virus is AAV1, AAV2, AAV3, AAV4, AAV5, AAV6, AAV7, AAV8, AAV9, AAV.rh74, AAV.rh8, AAV.rh10, AAV11, AAV12, AAV13, AAV-anc80, AAV-B1, AAV.PHP.EB, or AAVv66.

11. The adeno-associated virus of any one of claims 7-10, wherein the virus is AAV9.

12. A nanoparticle, extracellular vesicle, or exosome comprising the nucleic acid of any one of claims 1-6.

13. A composition comprising
(a) the nucleic acid of any one of claims 1-6;
(b) the adeno-associated virus of any one of claims 7-11; or
(c) the nanoparticle, extracellular vesicle, or exosome of claim 12; and
a pharmaceutically acceptable carrier.

14. A method of reducing, inhibiting, and/or interfering with expression of a Potassium Channel, Voltage Gated KQT-Like Subfamily Q, Member 3 (KCNQ3) gene or a variant thereof in a cell comprising contacting the cell with
(a) the nucleic acid of any one of claims 1-6;
(b) the adeno-associated virus of any one of claims 7-11;
(c) the nanoparticle, extracellular vesicle, or exosome of claim 12; and/or
(d) the composition of claim 13.

15. A method of treating a subject having a KCNQ3 mutation that results in the expression of a mutant or pathogenic form of KCNQ3 comprising administering to the subject an effective amount of
(a) the nucleic acid of any one of claims 1-6;
(b) the adeno-associated virus of any one of claims 7-11;
(c) the nanoparticle, extracellular vesicle, or exosome of claim 12; and/or
(d) the composition of claim 13.

16. The method of claim 15, wherein the mutation is a base substation, deletion, or insertion.

17. The method of claim 15 or 16, wherein the mutation is any one or more mutations in the KCNQ3 gene resulting in the substitution of R230C, R230H, R230S, and/or R227Q of the KCNQ3 polypeptide.

18. The method of any one of claims 15-17, wherein the subject suffers from seizures, an epileptic disease or disorder, an intellectual or developmental disability, autism, or an autism spectrum disorder associated with mutant or pathogenic KCNQ3 expression.

19. A method of treating or ameliorating a subject suffering from seizures, an epileptic disease or disorder, an intellectual or developmental disability, autism, or an autism spectrum disorder associated with mutant or pathogenic KCNQ3 expression comprising administering to the subject an effective amount of
(a) the nucleic acid of any one of claims 1-6;
(b) the adeno-associated virus of any one of claims 7-11;
(c) the nanoparticle, extracellular vesicle, or exosome of claim 12; and/or
(d) the composition of claim 13.

20. The method of claim 19, wherein the subject suffers from developmental and epileptic encephalopathy (DEE).

21. The method of claim 19 or 20, wherein the subject suffers from a mutation in the KCNQ3 gene, wherein the mutation is any one or more mutations in the KCNQ3 gene resulting in the substitution of R230C, R230H, R230S, and/or R227Q of the KCNQ3 polypeptide.

22. Use of
(a) the nucleic acid of any one of claims 1-6;
(b) the adeno-associated virus of any one of claims 7-11;
(c) the nanoparticle, extracellular vesicle, or exosome of claim 12; and/or
(d) the composition of claim 13
for the preparation of a medicament for reducing or inhibiting expression of a Potassium Channel, Voltage Gated KQT-Like Subfamily Q, Member 3 (KCNQ3) gene or a variant thereof in a cell.

23. Use of
(a) the nucleic acid of any one of claims 1-6;
(b) the adeno-associated virus of any one of claims 7-11;
(c) the nanoparticle, extracellular vesicle, or exosome of claim 12; and/or
(d) the composition of claim 13
for treating or ameliorating seizures, an epileptic disease or disorder, an intellectual or developmental disability, autism, or an autism spectrum disorder associated with mutant or pathogenic KCNQ3 expression.

24. Use of
(a) the nucleic acid of any one of claims 1-6;
(b) the adeno-associated virus of any one of claims 7-11;
(c) the nanoparticle, extracellular vesicle, or exosome of claim 12; and/or
(d) the composition of claim 13
for the preparation of a medicament for treating or ameliorating seizures, an epileptic disease or disorder, an intellectual or developmental disability, autism, or an autism spectrum disorder associated with mutant or pathogenic KCNQ3 expression.

25. The use of any one of claims 23 and 24, wherein the seizures, epileptic disease or disorder, the intellectual or developmental disability, autism, or the autism spectrum disorder associated with mutant or pathogenic KCNQ3 expression is developmental and epileptic encephalopathy (DEE).

26. The use of any one of claims 23 and 24, wherein the seizures, epileptic disease or disorder, the intellectual or developmental disability, autism, or the autism spectrum disorder associated with mutant or pathogenic KCNQ3 expression results from any one or more mutations in the KCNQ3 gene resulting in the substitution of R230C, R230H, R230S, and/or R227Q of the KCNQ3 polypeptide.

27. Use of
(a) the nucleic acid of any one of claims 1-6;
(b) the adeno-associated virus of any one of claims 7-11;
(c) the nanoparticle, extracellular vesicle, or exosome of claim 12; and/or
(d) the composition of claim 13
for reducing, inhibiting, and/or interfering with expression of a Potassium Channel, Voltage Gated KQT-Like Subfamily Q, Member 3 (KCNQ3) gene or a variant thereof in a cell.

28. The use of claim 27, wherein the cell is in a subject.

29. The use of claim 27 or 28, wherein the variant of the KCNQ3 results in the substitution of R230C, R230H, R230S, and/or R227Q of the KCNQ3 polypeptide.

30. The
(a) nucleic acid of any one of claims 1-6;
(b) adeno-associated virus (AAV) of any one of claims 7-11;
(c) nanoparticle, extracellular vesicle, or exosome of claim 12;
(d) composition of claim 13;
(e) method of any one of claims 14-21; or
(f) use of any one of claims 22-29,
wherein the nucleic acid, AAV, nanoparticle, extracellular vesicle, exosome, or composition, or medicament is formulated for intracerebroventricular injection, intrathecal injection, injection into the blood stream, aerosol administration, or oral administration.


SEQUENCE LISTING (45 sequences)

SEQ ID NO: 1 - DNA, 2619 bp, synthetic construct
  misc_feature 1..2619: note = Synthetic
  source 1..2619: mol_type = other DNA
atggggctcaaggcgcgcagggcggcgggggcggctggcggcggcggcgacgggggcggcggaggcggcggggcggctaacccagccggaggggacgcggcggcggccggcgacgaggagcggaaagtggggctggcgcccggcgacgtggagcaagtcaccttggcgctcggggccggagccgacaaagacgggaccctgctgctggagggcggcggccgcgacgaggggcagcggaggaccccgcagggcatcgggctcctggccaagaccccgctgagccgcccagtcaagagaaacaacgccaagtaccggcgcatccaaactttgatctacgacgccctggagagaccgcggggctgggcgctgctttaccacgcgttggtgttcctgattgtcctggggtgcttgattctggctgtcctgaccacattcaaggagtatgagactgtctcgggagactggcttctgttactggagacatttgctattttcatctttggagccgagtttgctttgaggatctgggctgctggatgttgctgccgatacaaaggctggcggggccgactgaagtttgccaggaagcccctgtgcatgttggacatctttgtgctgattgcctctgtgccagtggttgctgtgggaaaccaaggcaatgttctggccacctccctgcgaagcctgcgcttcctgcagatcctgcgcatgctgcggatggaccggagaggtggcacctggaagcttctgggctcagccatctgtgcccacagcaaagaactcatcacggcctggtacatcggtttcctgacactcatcctttcttcatttcttgtctacctggttgagaaagacgtcccagaggtggatgcacaaggagaggagatgaaagaggagtttgagacctatgcagatgccctgtggtggggcctgatcacactggccaccattggctatggagacaagacacccaaaacgtgggaaggccgtctgattgccgccaccttttccttaattggcgtctccttttttgcccttccagcgggcatcctggggtccgggctggccctcaaggtgcaggagcaacaccgtcagaagcactttgagaaaaggaggaagccagctgctgagctcattcaggctgcctggaggtattatgctaccaaccccaacaggattgacctggtggcgacatggagattttatgaatcagtcgtctcttttcctttcttcaggaaagaacagctggaggcagcatccagccaaaagctgggtctcttggatcgggttcgcctttctaatcctcgtggtagcaatactaaaggaaagctatttacccctctgaatgtagatgccatagaagaaagtccttctaaagaaccaaagcctgttggcttaaacaataaagagcgtttccgcacggccttccgcatgaaagcctacgctttctggcagagttctgaagatgccgggacaggtgaccccatggcggaagacaggggctatgggaatgacttccccatcgaagacatgatccccaccctgaaggccgccatccgagccgtcagaattctacaattccgtctctataaaaaaaaattcaaggagactttgaggccttacgatgtgaaggatgtgattgagcagtattctgccgggcatctcgacatgctttccaggataaagtaccttcagacgagaatagatatgattttcacccctggacctccctccacgccaaaacacaagaagtctcagaaagggtcagcattcaccttcccatcccagcaatctcccaggaatgaaccatatgtagccagaccatccacatcagaaatcgaagaccaaagcatgatggggaagtttgtaaaagttgaaagacaggttcaggacatggggaagaagctggacttcctcgtggatatgcacatgcaacacatggaacggttgcaggtgcaggtcacggagtattacccaaccaagggcacctcctcgccagctgaagcagagaagaaggaggacaacaggtattccgatttgaaaaccatcatctgcaactattctgagacaggccccccggaaccaccctacagcttccaccaggtgaccattgacaaagtcagcccctatgggttttttgcacatgaccctgtgaacctgccccgagggggacccagttctggaaaggttcaggcaactcctccttcctcagcaacaacgtatgtggagaggcccacggtcctgcctatcttgactcttctcgactcccgagtgagctgccactcccaggctgacctgcagggcccctactcggaccgaatctccccccggcagagacgtagcatcacgcgagacagtgacacacctctgtccctgatgtcggtcaaccacgaggagctggagaggtctccaagtggcttcagcatctcccaggacagagatgattatgtgttcggccccaatggggggtcgagctggatgagggagaagcggtacctcgccgagggtgagacggacacagacacggaccccttcacgcccagcggctccatgcctctgtcgtccacaggggatgggatttctgattcagtatggaccccttccaataagcccatttaa

SEQ ID NO: 2 - protein, 872 aa, synthetic construct
  REGION 1..872: note = Synthetic
MGLKARRAAGAAGGGGDGGGGGGGAANPAGGDAAAAGDEERKVGLAPGDVEQVTLALGAGADKDGTLLLEGGGRDEGQRRTPQGIGLLAKTPLSRPVKRNNAKYRRIQTLIYDALERPRGWALLYHALVFLIVLGCLILAVLTTFKEYETVSGDWLLLLETFAIFIFGAEFALRIWAAGCCCRYKGWRGRLKFARKPLCMLDIFVLIASVPVVAVGNQGNVLATSLRSLRFLQILRMLRMDRRGGTWKLLGSAICAHSKELITAWYIGFLTLILSSFLVYLVEKDVPEVDAQGEEMKEEFETYADALWWGLITLATIGYGDKTPKTWEGRLIAATFSLIGVSFFALPAGILGSGLALKVQEQHRQKHFEKRRKPAAELIQAAWRYYATNPNRIDLVATWRFYESVVSFPFFRKEQLEAASSQKLGLLDRVRLSNPRGSNTKGKLFTPLNVDAIEESPSKEPKPVGLNNKERFRTAFRMKAYAFWQSSEDAGTGDPMAEDRGYGNDFPIEDMIPTLKAAIRAVRILQFRLYKKKFKETLRPYDVKDVIEQYSAGHLDMLSRIKYLQTRIDMIFTPGPPSTPKHKKSQKGSAFTFPSQQSPRNEPYVARPSTSEIEDQSMMGKFVKVERQVQDMGKKLDFLVDMHMQHMERLQVQVTEYYPTKGTSSPAEAEKKEDNRYSDLKTIICNYSETGPPEPPYSFHQVTIDKVSPYGFFAHDPVNLPRGGPSSGKVQATPPSSATTYVERPTVLPILTLLDSRVSCHSQADLQGPYSDRISPRQRRSITRDSDTPLSLMSVNHEELERSPSGFSISQDRDDYVFGPNGGSSWMREKRYLAEGETDTDTDPFTPSGSMPLSSTGDGISDSVWTPSNKPI

SEQ ID NO: 3 - DNA, 22 bp, synthetic construct
  misc_feature 1..22: note = Synthetic
  source 1..22: mol_type = other DNA
tagatcaaagtttggatgcgcc

SEQ ID NO: 4 - DNA, 22 bp, synthetic construct
  misc_feature 1..22: note = Synthetic
  source 1..22: mol_type = other DNA
aacatgcacaggggcttcctgg

SEQ ID NO: 5 - DNA, 22 bp, synthetic construct
  misc_feature 1..22: note = Synthetic
  source 1..22: mol_type = other DNA
atgagttctttgctgtgggcac

SEQ ID NO: 6 - DNA, 22 bp, synthetic construct
  misc_feature 1..22: note = Synthetic
  source 1..22: mol_type = other DNA
tgatgagttctttgctgtgggc

SEQ ID NO: 7 - DNA, 22 bp, synthetic construct
  misc_feature 1..22: note = Synthetic
  source 1..22: mol_type = other DNA
tgaagaaaggatgagtgtcagg

SEQ ID NO: 8 - DNA, 22 bp, synthetic construct
  misc_feature 1..22: note = Synthetic
  source 1..22: mol_type = other DNA
aatggtggccagtgtgatcagg

SEQ ID NO: 9 - DNA, 22 bp, synthetic construct
  misc_feature 1..22: note = Synthetic
  source 1..22: mol_type = other DNA
agaaagcgtaggctttcatgcg

SEQ ID NO: 10 - DNA, 477 bp, synthetic construct
  misc_feature 1..477: note = Synthetic
  source 1..477: mol_type = other DNA
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgagcgcatccaaactttgatctactgtaaagccacagatgggtagatcaaagtttggatgcgcctgcctactagagcggccgccacagcggggagatccagacatgataagatacatttttt

SEQ ID NO: 11 - DNA, 477 bp, synthetic construct
  misc_feature 1..477: note = Synthetic
  source 1..477: mol_type = other DNA
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgacaggaagcccctgtgcatgttctgtaaagccacagatgggaacatgcacaggggcttcctggtgcctactagagcggccgccacagcggggagatccagacatgataagatacatttttt

SEQ ID NO: 12 - DNA, 477 bp, synthetic construct
  misc_feature 1..477: note = Synthetic
  source 1..477: mol_type = other DNA
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgatgcccacagcaaagaactcatctgtaaagccacagatgggatgagttctttgctgtgggcactgcctactagagcggccgccacagcggggagatccagacatgataagatacatttttt

SEQ ID NO: 13 - DNA, 477 bp, synthetic construct
  misc_feature 1..477: note = Synthetic
  source 1..477: mol_type = other DNA
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgacccacagcaaagaactcatcactgtaaagccacagatgggtgatgagttctttgctgtgggctgcctactagagcggccgccacagcggggagatccagacatgataagatacatttttt

SEQ ID NO: 14 - DNA, 477 bp, synthetic construct
  misc_feature 1..477: note = Synthetic
  source 1..477: mol_type = other DNA
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgactgacactcatcctttcttcactgtaaagccacagatgggtgaagaaaggatgagtgtcaggtgcctactagagcggccgccacagcggggagatccagacatgataagatacatttttt

SEQ ID NO: 15 - DNA, 477 bp, synthetic construct
  misc_feature 1..477: note = Synthetic
  source 1..477: mol_type = other DNA
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgactgatcacactggccaccattctgtaaagccacagatgggaatggtggccagtgtgatcaggtgcctactagagcggccgccacagcggggagatccagacatgataagatacatttttt

SEQ ID NO: 16 - DNA, 477 bp, synthetic construct
  misc_feature 1..477: note = Synthetic
  source 1..477: mol_type = other DNA
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgagcatgaaagcctacgctttctctgtaaagccacagatgggagaaagcgtaggctttcatgcgtgcctactagagcggccgccacagcggggagatccagacatgataagatacatttttt

SEQ ID NO: 17 - RNA, 22 nt, synthetic construct
  misc_feature 1..22: note = Synthetic
  source 1..22: mol_type = other RNA
  modified_base 1: mod_base = OTHER; note = Uracil
  modified_base 5: mod_base = OTHER; note = Uracil
  modified_base 11..13: mod_base = OTHER; note = Uracil
  modified_base 17: mod_base = OTHER; note = Uracil
tagatcaaagtttggatgcgcc

SEQ ID NO: 18 - RNA, 22 nt, synthetic construct
  misc_feature 1..22: note = Synthetic
  source 1..22: mol_type = other RNA
  modified_base 5: mod_base = OTHER; note = Uracil
  modified_base 16..17: mod_base = OTHER; note = Uracil
  modified_base 20: mod_base = OTHER; note = Uracil
aacatgcacaggggcttcctgg

SEQ ID NO: 19 - RNA, 22 nt, synthetic construct
  misc_feature 1..22: note = Synthetic
  source 1..22: mol_type = other RNA
  modified_base 2: mod_base = OTHER; note = Uracil
  modified_base 6..7: mod_base = OTHER; note = Uracil
  modified_base 9..11: mod_base = OTHER; note = Uracil
  modified_base 14: mod_base = OTHER; note = Uracil
  modified_base 16: mod_base = OTHER; note = Uracil
atgagttctttgctgtgggcac

SEQ ID NO: 20 - RNA, 22 nt, synthetic construct
  misc_feature 1..22: note = Synthetic
  source 1..22: mol_type = other RNA
  modified_base 1: mod_base = OTHER; note = Uracil
  modified_base 4: mod_base = OTHER; note = Uracil
  modified_base 8..9: mod_base = OTHER; note = Uracil
  modified_base 11..13: mod_base = OTHER; note = Uracil
  modified_base 16: mod_base = OTHER; note = Uracil
  modified_base 18: mod_base = OTHER; note = Uracil
tgatgagttctttgctgtgggc

SEQ ID NO: 21 - RNA, 22 nt, synthetic construct
  misc_feature 1..22: note = Synthetic
  source 1..22: mol_type = other RNA
  modified_base 1: mod_base = OTHER; note = Uracil
  modified_base 12: mod_base = OTHER; note = Uracil
  modified_base 16: mod_base = OTHER; note = Uracil
  modified_base 18: mod_base = OTHER; note = Uracil
tgaagaaaggatgagtgtcagg

SEQ ID NO: 22 - RNA, 22 nt, synthetic construct
  misc_feature 1..22: note = Synthetic
  source 1..22: mol_type = other RNA
  modified_base 3: mod_base = OTHER; note = Uracil
  modified_base 6: mod_base = OTHER; note = Uracil
  modified_base 13: mod_base = OTHER; note = Uracil
  modified_base 15: mod_base = OTHER; note = Uracil
  modified_base 18: mod_base = OTHER; note = Uracil
aatggtggccagtgtgatcagg

SEQ ID NO: 23 - RNA, 22 nt, synthetic construct
  misc_feature 1..22: note = Synthetic
  source 1..22: mol_type = other RNA
  modified_base 9: mod_base = OTHER; note = Uracil
  modified_base 14..16: mod_base = OTHER; note = Uracil
  modified_base 19: mod_base = OTHER; note = Uracil
agaaagcgtaggctttcatgcg

SEQ ID NO: 24 - RNA, 22 nt, synthetic construct
  misc_feature 1..22: note = Synthetic
  source 1..22: mol_type = other RNA
  modified_base 7: mod_base = OTHER; note = Uracil
  modified_base 14..16: mod_base = OTHER; note = Uracil
  modified_base 19: mod_base = OTHER; note = Uracil
  modified_base 21: mod_base = OTHER; note = Uracil
ggcgcatccaaactttgatcta

SEQ ID NO: 25 - RNA, 22 nt, synthetic construct
  misc_feature 1..22: note = Synthetic
  source 1..22: mol_type = other RNA
  modified_base 13: mod_base = OTHER; note = Uracil
  modified_base 15: mod_base = OTHER; note = Uracil
  modified_base 19: mod_base = OTHER; note = Uracil
  modified_base 21..22: mod_base = OTHER; note = Uracil
ccaggaagcccctgtgcatgtt

SEQ ID NO: 26 - RNA, 22 nt, synthetic construct
  misc_feature 1..22: note = Synthetic
  source 1..22: mol_type = other RNA
  modified_base 2: mod_base = OTHER; note = Uracil
  modified_base 19: mod_base = OTHER; note = Uracil
  modified_base 22: mod_base = OTHER; note = Uracil
gtgcccacagcaaagaactcat

SEQ ID NO: 27 - RNA, 22 nt, synthetic construct
  misc_feature 1..22: note = Synthetic
  source 1..22: mol_type = other RNA
  modified_base 17: mod_base = OTHER; note = Uracil
  modified_base 20: mod_base = OTHER; note = Uracil
gcccacagcaaagaactcatca

SEQ ID NO: 28 - RNA, 22 nt, synthetic construct
  misc_feature 1..22: note = Synthetic
  source 1..22: mol_type = other RNA
  modified_base 3: mod_base = OTHER; note = Uracil
  modified_base 9: mod_base = OTHER; note = Uracil
  modified_base 12: mod_base = OTHER; note = Uracil
  modified_base 15..17: mod_base = OTHER; note = Uracil
  modified_base 19..20: mod_base = OTHER; note = Uracil
cctgacactcatcctttcttca

SEQ ID NO: 29 - RNA, 22 nt, synthetic construct
  misc_feature 1..22: note = Synthetic
  source 1..22: mol_type = other RNA
  modified_base 3: mod_base = OTHER; note = Uracil
  modified_base 6: mod_base = OTHER; note = Uracil
  modified_base 12: mod_base = OTHER; note = Uracil
  modified_base 21..22: mod_base = OTHER; note = Uracil
cctgatcacactggccaccatt

SEQ ID NO: 30 - RNA, 22 nt, synthetic construct
  misc_feature 1..22: note = Synthetic
  source 1..22: mol_type = other RNA
  modified_base 5: mod_base = OTHER; note = Uracil
  modified_base 13: mod_base = OTHER; note = Uracil
  modified_base 18..20: mod_base = OTHER; note = Uracil
  modified_base 22: mod_base = OTHER; note = Uracil
cgcatgaaagcctacgctttct

SEQ ID NO: 31 - DNA, 86 bp, synthetic construct
  misc_feature 1..86: note = Synthetic
  source 1..86: mol_type = other DNA
ctcgagtgagcgagcgcatccaaactttgatctactgtaaagccacagatgggtagatcaaagtttggatgcgcctgcctactaga

SEQ ID NO: 32 - DNA, 86 bp, synthetic construct
  misc_feature 1..86: note = Synthetic
  source 1..86: mol_type = other DNA
ctcgagtgagcgacaggaagcccctgtgcatgttctgtaaagccacagatgggaacatgcacaggggcttcctggtgcctactaga

SEQ ID NO: 33 - DNA, 86 bp, synthetic construct
  misc_feature 1..86: note = Synthetic
  source 1..86: mol_type = other DNA
ctcgagtgagcgatgcccacagcaaagaactcatctgtaaagccacagatgggatgagttctttgctgtgggcactgcctactaga

SEQ ID NO: 34 - DNA, 86 bp, synthetic construct
  misc_feature 1..86: note = Synthetic
  source 1..86: mol_type = other DNA
ctcgagtgagcgacccacagcaaagaactcatcactgtaaagccacagatgggtgatgagttctttgctgtgggctgcctactaga

SEQ ID NO: 35 - DNA, 86 bp, synthetic construct
  misc_feature 1..86: note = Synthetic
  source 1..86: mol_type = other DNA
ctcgagtgagcgactgacactcatcctttcttcactgtaaagccacagatgggtgaagaaaggatgagtgtcaggtgcctactaga

SEQ ID NO: 36 - DNA, 86 bp, synthetic construct
  misc_feature 1..86: note = Synthetic
  source 1..86: mol_type = other DNA
ctcgagtgagcgactgatcacactggccaccattctgtaaagccacagatgggaatggtggccagtgtgatcaggtgcctactaga

SEQ ID NO: 37 - DNA, 86 bp, synthetic construct
  misc_feature 1..86: note = Synthetic
  source 1..86: mol_type = other DNA
ctcgagtgagcgagcatgaaagcctacgctttctctgtaaagccacagatgggagaaagcgtaggctttcatgcgtgcctactaga

SEQ ID NO: 38 - RNA, 20 nt, synthetic construct
  misc_feature 1..20: note = Synthetic
  source 1..20: mol_type = other RNA
  modified_base 7: mod_base = OTHER; note = Uracil
  modified_base 9: mod_base = OTHER; note = Uracil
gcaggatctgcaggaagcga

SEQ ID NO: 39 - DNA, 21 bp, synthetic construct
  misc_feature 1..21: note = Synthetic
  source 1..21: mol_type = other DNA
caccgtcagaagcactttgag

SEQ ID NO: 40 - DNA, 23 bp, synthetic construct
  misc_feature 1..23: note = Synthetic
  source 1..23: mol_type = other DNA
cctttagtattgctaccacgagg

SEQ ID NO: 41 - DNA, 20 bp, synthetic construct
  misc_feature 1..20: note = Synthetic
  source 1..20: mol_type = other DNA
ggctgtattcccctccatcg

SEQ ID NO: 42 - DNA, 19 bp, synthetic construct
  misc_feature 1..19: note = Synthetic
  source 1..19: mol_type = other DNA
ccaggtaacaatgccatgt

SEQ ID NO: 43 - DNA, 20 bp, synthetic construct
  misc_feature 1..20: note = Synthetic
  source 1..20: mol_type = other DNA
acgtaaacggccacaagttc

SEQ ID NO: 44 - DNA, 20 bp, synthetic construct
  misc_feature 1..20: note = Synthetic
  source 1..20: mol_type = other DNA
ctgggtgctcaggtagtggt

SEQ ID NO: 45 - skipped